(19) United States
(12) Patent Application Publication
(10) Pub. No.: US 2024/0315657 A1
(43) Pub. Date: Sep. 26, 2024

(54) METHODS AND SYSTEMS FOR DATA CORRECTION

(71) Applicant: WUHAN UNITED IMAGING LIFE SCIENCE INSTRUMENT CO., LTD., Wuhan (CN)

(72) Inventors: Tan XU, Wuhan (CN); Wenting XU, Wuhan (CN); Wei YANG, Wuhan (CN)

(73) Assignee: WUHAN UNITED IMAGING LIFE SCIENCE INSTRUMENT CO., LTD., Wuhan (CN)

(21) Appl. No.: 18/736,514

(22) Filed: Jun. 6, 2024

Related U.S. Application Data
(63) Continuation of application No. PCT/CN2022/102081, filed on Jun. 28, 2022.

(30) Foreign Application Priority Data
Dec. 23, 2021 (CN) 202111594782.2

(51) Int. Cl. A61B 6/00; A61B 6/42; A61B 6/58

(52) U.S. Cl. CPC A61B 6/5258; A61B 6/4233; A61B 6/583; A61B 6/563

(57) ABSTRACT

The embodiments of the present disclosure provide a method and system for data correction. The method is applied to a medical scanning device including a ray emitting device and a detector. The detector may include a plurality of detector pixel units. The method for data correction may include obtaining spatial positions of the plurality of detector pixel units; determining cosine correction data based on the spatial positions of the plurality of detector pixel units and a spatial position of a focal point of the ray emitting device; determining response data to be corrected of the detector; determining target data by correcting the response data to be corrected using the cosine correction data; determining one or more correction coefficients corresponding to the plurality of detector pixel units based on the target data; and correcting response data of a subject to be detected based on the one or more correction coefficients.

### CROSS-REFERENCE RELATED TO APPLICATIONS

This application is a Continuation of International Application No. PCT/CN2022/102081, filed on Jun. 28, 2022, which claims priority to Chinese application No. 202111594782.2, filed on Dec. 23, 2021, the entire contents of each of which are incorporated herein by reference.

### TECHNICAL FILED

The present disclosure relates to the field of medical imaging computing, and in particular, to methods and systems for data correction.

### BACKGROUND

With the development of science and technology, photon counting detectors have gradually been widely used in medical imaging. However, photon counting detectors suffer from the problem of uneven response among pixels, which can lead to artifacts in the reconstructed images. In conventional techniques, the response data of detector pixel units is corrected by employing the flat field correction technique to improve the uniformity of the response of the detector pixel units, thereby eliminating the artifacts in the reconstructed images. However, the flat field correction technique has a poor effect on improving the response uniformity of the detector.

Therefore, there is a need to provide a method and system for data correction to reduce artifacts in reconstructed images.

### SUMMARY

According to one or more embodiments of the present disclosure, a method for data correction is provided, the method may be applied to a medical scanning device, the medical scanning device may include a ray emitting device and a detector, the detector may include a plurality of detector pixel units. The method may include: obtaining spatial positions of the plurality of detector pixel units; determining cosine correction data based on the spatial positions of the plurality of detector pixel units and a spatial position of a focal point of the ray emitting device; determining response data to be corrected of the detector; determining target data by correcting the response data to be corrected using the cosine correction data; determining one or more correction coefficients corresponding to the plurality of detector pixel units based on the target data; and correcting response data of a subject to be detected based on the one or more correction coefficients.

In some embodiments, the determining one or more correction coefficients corresponding to the plurality of detector pixel units based on the target data may include: for each row of the plurality of detector pixel units of the detector in a preset direction, determining a mean value of target data corresponding to the row of the plurality of detector pixel units, designating the mean value as correction data of the row of the plurality of detector pixel units; and determining the one or more correction coefficients based on correction data of all the plurality of detector pixel units.

In some embodiments, the response data to be corrected may include data of the detector in response to homogeneous plates of different thicknesses.

In some embodiments, the determining the one or more correction coefficients based on correction data of all the plurality of detector pixel units may include: establishing a linear relationship between the target data and the correction data of the all the plurality of detector pixel units; and determining the one or more correction coefficients by solving the linear relationship between the target data and the correction data of the all the plurality of detector pixel units based on the correction data of the all the plurality of detector pixel units corresponding to the homogeneous plates of different thicknesses.

In some embodiments, the obtaining spatial positions of the plurality of detector pixel units may include: obtaining a projection image of a testing phantom on the detector; determining a response center point of the detector based on the projection image; and determining three-dimensional coordinates of the plurality of detector pixel units based on the response center point, distances between the plurality of detector pixel units, and a distance between the focal point of the ray emitting device and the response center point.

In some embodiments, the determining cosine correction data based on the spatial positions of the plurality of detector pixel units and a spatial position of a focal point of the ray emitting device may include: for each of the plurality of detector pixel units, obtaining the cosine correction data by determining cosine of an angle between a first connecting line and a second connecting line according to the three-dimensional coordinates of the plurality of detector pixel units and coordinates of the focal point, wherein the first connecting line may be a connecting line between the detector pixel unit and the focal point of the ray emitting device, the second connecting line may be a connecting line between the focal point of the ray emitting device and a vertical point on the detector, and the second connecting line may be perpendicular to the detector.

In some embodiments, the vertical point may be the response center point.

In some embodiments, the determining response data to be corrected of the detector may include: obtaining phantom data and air data by the detector, wherein the phantom data may be the response data of the detector when the testing phantom is disposed between the ray emitting device and the detector, and the air data may be the response data of the detector when the testing phantom is not disposed between the ray emitting device and the detector; and determining the response data to be corrected based on the air data and phantom data.

In some embodiments, the testing phantom may include homogeneous plates of at least two thicknesses.

According to one or more embodiments of the present disclosure, a data correction system is provided, the system may include: an obtaining module configured to obtain spatial positions of a plurality of detector pixel units, and determine cosine correction data based on the spatial positions of the plurality of detector pixel units and a spatial position of a focal point of a ray emitting device; a determination module configured to determine response data to be corrected of the detector, and obtain target data by correcting the response data to be corrected using the cosine correction data; and a correction module configured to determine one or more correction coefficients corresponding to the plurality of detector pixel units based on the target data, and correct the response data of a subject to be detected based on the one or more correction coefficients.

In some embodiments, the correction module may be further configured to: for each row of the plurality of detector pixel units of the detector in a preset direction, determine a mean value of target data corresponding to the row of the plurality of detector pixel units, designate the mean value as correction data of the row of the plurality of detector pixel units; and determine the one or more correction coefficients based on the correction data of all the plurality of detector pixel units.

In some embodiments, the response data to be corrected may include data of the detector in response to homogeneous plates of different thicknesses.

In some embodiments, the correction module may be further configured to: establish a linear relationship between the target data and the correction data of the all the plurality of detector pixel units; and determine the one or more correction coefficients by solving the linear relationship between the target data and the correction data of the all the plurality of detector pixel units based on the correction data of the all the plurality of detector pixel units corresponding to the homogeneous plates of different thicknesses.

In some embodiments, the obtaining module may be further configured to: obtain a projection image of a testing phantom on the detector; determine a response center point of the detector based on the projection image; and determine three-dimensional coordinates of the plurality of detector pixel units based on the response center point, distances between the plurality of detector pixel units, and a distance between the focal point of the ray emitting device and the response center point.

In some embodiments, the obtaining module may be further configured to: for each of the plurality of detector pixel units, obtain the cosine correction data by determining cosine of an angle between a first connecting line and a second connecting line according to the three-dimensional coordinates of the plurality of detector pixel units and coordinates of the focal point, wherein the first connecting line may be a connecting line between the detector pixel unit and the focal point of the ray emitting device, the second connecting line may be a connecting line between the focal point of the ray emitting device and a vertical point on the detector, and the second connecting line may be perpendicular to the detector.

In some embodiments, the vertical point may be the response center point.

In some embodiments, the determination module may be further configured to: obtain the phantom data and air data by the detector, wherein the phantom data may be the response data of the detector when a testing phantom is disposed between the ray emitting device and the detector, and the air data is response data of the detector when the testing phantom is not disposed between the ray emitting device and the detector; and determine the response data to be corrected based on the air data and phantom data.

In some embodiments, the testing phantom may include homogeneous plates of at least two thicknesses.

In some embodiments, the system further may include a distribution determination module configured to: determine a plurality of combinations of homogeneous plates, the combinations of homogeneous plates may include homogeneous plates of at least two thicknesses, the homogeneous plates of at least two thicknesses may include at least two base substances; obtain air correction data of the detector in response to each of the homogeneous plates of different thicknesses; determine one or more decomposition coefficients of the homogeneous plates of at least two thicknesses for each material based on the air correction data of the detector in response to the homogeneous plates of different thicknesses; and determine a distribution of the base substances of the subject to be detected based on the response data of the subject to be detected and the one or more decomposition coefficients.

In some embodiments, the system may further include an energy determination module configured to: determine one or more effective attenuation coefficients of the homogeneous plates based on the data correction in response to the homogeneous plates of different thicknesses; and determine an effective energy of the ray emitting device or the detector based on the one or more effective attenuation coefficients of the homogeneous plates.

According to one or more embodiments of the present disclosure, a device for data correction is provided, the device may include a processor, and the processor may be configured to perform the method for data correction.

According to one or more embodiments of the present disclosure, a non-transitory computer-readable storage medium is provided, the storage medium may store computer instructions, and the computer, when reading the computer instructions in the storage medium, may perform the method for data correction.

According to one of the embodiments of the present disclosure, a method or system for data correction is provided. The method includes obtaining the spatial positions of the plurality of detector pixel units; determining the cosine correction data based on the spatial positions of the plurality of detector pixel units and the spatial position of the focal point of the ray emitting device; determining the response data to be corrected of the detector; obtaining the target data by correcting the response data to be corrected using the cosine correction data; determining the one or more correction coefficients corresponding to the plurality of detector pixel units based on the target data; and correcting the response data of a subject to be detected based on the one or more correction coefficients, such that correction coefficients for correcting inhomogeneity of the plurality of detector pixel units can be obtained. The response data of the subject to be detected may be corrected more accurately through the one or more correction coefficients such that the effect of correcting the inhomogeneity of the plurality of detector pixel units can be improved, thereby making a reconstruction image of the subject to be detected have lower noise and fewer artifacts.

### BRIEF DESCRIPTION OF THE DRAWING

The present disclosure may be further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 is a schematic diagram illustrating an application scenario of a system for data correction according to some embodiments of the present disclosure;

FIG. 2 is a block diagram illustrating a system for data correction according to some embodiments of the present disclosure;

FIG. 3 is a flowchart illustrating an exemplary process for data correction according to some embodiments of the present disclosure;

FIG. 4 is a schematic diagram illustrating a three-dimensional Cartesian coordinate system in which three-dimensional coordinates of detector pixel units are located according to some embodiments of the present disclosure;

FIG. 5 is a flowchart illustrating an exemplary determination of correction coefficients corresponding to a plurality of detector pixel units according to each row of the plurality of detector pixel units according to some embodiments of the present disclosure;

FIG. 6 is a flowchart illustrating an exemplary process for analyzing a distribution of base substances of a subject to be detected according to some embodiments of the present disclosure;

FIG. 7 is a flowchart illustrating an exemplary process for determining effective energy according to some embodiments of the present disclosure;

FIG. 8 is a flowchart illustrating an exemplary process for determining one or more correction coefficients based on correction data of all the plurality of detector pixel units according to some embodiments of the present disclosure;

FIG. 9 is a schematic diagram illustrating an application environment of a method for data correction according to some embodiments of the present disclosure;

FIG. 10 is a schematic diagram illustrating a structure of a ray emitting device according to some embodiments of the present disclosure;

FIG. 11 is a flow schematic diagram illustrating operations of a method for data correction according to some embodiments of the present disclosure;

FIG. 12 is a schematic diagram illustrating a cosine correction matrix according to some embodiments of the present disclosure;

FIG. 13 is a schematic diagram illustrating a process for obtaining three-dimensional coordinates of detector pixel units according to some embodiments of the present disclosure;

FIG. 14 is a schematic diagram illustrating a process for obtaining response data to be corrected of a detector according to some embodiments of the present disclosure;

FIG. 15 is a schematic diagram illustrating response data to be corrected before and after correction corresponding to a 10 mm thick polymethylmethacrylate plate according to some embodiments of the present disclosure;

FIG. 16 is a schematic diagram illustrating response data to be corrected before and after correction corresponding to a 100 mm thick polymethylmethacrylate plate according to some embodiments of the present disclosure;

FIG. 17 is a schematic diagram illustrating a sine diagram of response data of water film before correction according to some embodiments of the present disclosure;

FIG. 18 is a schematic diagram illustrating a sine diagram of response data of water film after correction according to some embodiments of the present disclosure; and

FIG. 19 is a schematic diagram illustrating an internal structure of a computer device according to some embodiments of the present disclosure.

### DETAILED DESCRIPTION

In order to illustrate the technical solutions related to the embodiments of the present disclosure, a brief introduction of the drawings referred to in the description of the embodiments is provided below. Obviously, the drawings described below are only some examples or embodiments of the present disclosure. Those skilled in the art, without further creative efforts, may apply the present disclosure to other similar scenarios according to these drawings. Unless apparent from the locale or otherwise stated, like reference numerals represent similar structures or operations throughout the several views of the drawings.

It will be understood that the terms “system,” “device,” “unit,” and/or “module” used herein are one method to distinguish different components, elements, parts, sections, or assemblies of different levels in ascending order. However, the terms may be displaced by another expression if they achieve the same purpose.

As used in the disclosure and the appended claims, the singular forms “a,” “an,” and/or “the” may include plural forms unless the context clearly indicates otherwise. In general, the terms “comprise,” “comprises,” and/or “comprising,” “include,” “includes,” and/or “including,” merely prompt to include steps and elements that have been clearly identified, and these steps and elements do not constitute an exclusive listing. The methods or devices may also include other steps or elements.

The flowcharts used in the present disclosure illustrate operations that systems implement according to some embodiments in the present disclosure. It is to be expressly understood, the operations of the flowchart may be implemented not in order. Conversely, the operations may be implemented in an inverted order, or simultaneously. Moreover, one or more other operations may be added to the flowcharts. One or more operations may be removed from the flowcharts.

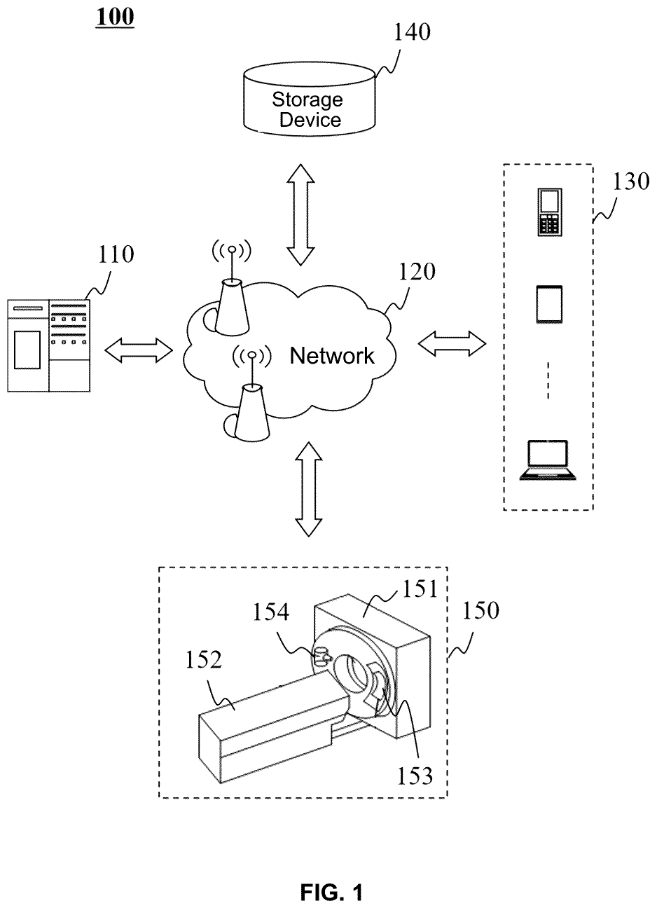

FIG. 1 is a schematic diagram illustrating an application scenario 100 of a system for data correction according to some embodiments of the present disclosure.

As shown in FIG. 1, in some embodiments, the application scenario 100 may include a processor 110, a network 120, a user terminal 130, a storage device 140, and a medical scanning device 150. The application scenario 100 may quickly and accurately correct response data of a subject to be detected by implementing operations and/or processes disclosed in the present disclosure.

The processor 110 may be configured to process data and/or information from at least one component of the application scenario 100 or an external data source (e.g., a cloud data center). The processor 110 may access the data and/or information from the user terminal 130, the storage device 140, and the medical scanning device 150 via the network 120. The processor 110 may be directly connected to the user terminal 130, the storage device 140, and the medical scanning device 150 to access the information and/or data. For example, the processor 110 may obtain spatial positions of a plurality of detector pixel units (referred to as the detector pixel units below). The processor 110 may process the obtained data and/or information. For example, the processor 110 may determine cosine correction data based on the obtained spatial positions of the plurality of detector pixel units and a focal point of a ray emitting device, obtain target data by correcting the obtained response data to be corrected using the cosine correction data, determine one or more correction coefficients corresponding to the detector pixel units based on the target data, and correct the response data of a subject to be detected based on the one or more correction coefficients. In some embodiments, the processor 110 may be a single server or a server group. The processor 110 may be local or remote.

The network 120 may include any suitable network that provides information and/or data exchange capable of facilitating the exchange of information and/or data for the application scenario 100. In some embodiments, the information and/or data may be exchanged between one or more components of the application scenario 100 (e.g., the processor 110, the user terminal 130, the storage device 140, and the medical scanning device 150) via the network 120.

In some embodiments, the network 120 may be any one or more of a wired network or a wireless network. In some embodiments, the network 120 may include one or more network access points. For example, the network 120 may include wired or wireless network access points, such as a base station and/or a network switching point, through which one or more components of the application scenario 100 may be connected to the network 120 to exchange the data and/or information.

The user terminal 130 refers to one or more terminals or software used by a user. In some embodiments, the user terminal 130 may refer to a terminal or software used by a healthcare worker (e.g., a nurse practitioner, a doctor, etc.). In some embodiments, the user terminal 130 may include, but is not limited to, a smart phone, a tablet computer, a laptop computer, a desktop computer, or the like. In some embodiments, the user terminal 130 may interact with other components in the application scenario 100 via the network 120. For example, the user terminal 130 may send one or more control instructions to the processor 110 to control the processor 110 to correct the response data of the subject to be detected.

The storage device 140 may be configured to store data, instructions, and/or any other information. In some embodiments, the storage device 140 may store data and/or information obtained from the processor 110, the user terminal 130, the storage device 140, the medical scanning device 150, and the like. For example, the storage device 140 may store the spatial positions of the obtained plurality of detector pixel units. In some embodiments, the storage device 140 may include a mass memory, a removable memory, etc., or any combination thereof.

The medical scanning device 150 is a device configured to obtain a medical image of the user. In some embodiments, the medical scanning device 150 may scan a subject, obtain scanning data, and generate a medical image of the user. The subject may be the whole of the subject to be detected or a portion thereof. The subject to be detected may include a living organism such as a human body, an animal, and the like. For example, the subject may include an organ, a tissue, a lesion site, a tumor site, or any combination thereof. Specifically, the subject may be the head, the chest, the abdomen, the heart, the liver, an upper limb, a lower limb, etc., or any combination thereof. In some embodiments, the medical scanning device 150 may be a single device or a device group. Specifically, the medical scanning device 150 may be a medical imaging system, such as a positron emission tomography (PET) device, a single photon emission computed tomography (SPECT) device, a computed tomography (CT) device, a magnetic resonance imaging (MRI) device, and the like. Further, the medical imaging systems may be used individually or in combination. For example, a positron emission tomography-computed tomography (PETCT) device, a positron emission tomography-magnetic resonance imaging (PETMRI) device, or a single photon emission computed tomography-magnetic resonance imaging system (SPECT/MRI) device, and the like.

In some embodiments, the medical scanning device 150 may include a cavity 151, a bed frame 152, an operation control computer device, and an image generator. The cavity 151 may accommodate components within the cavity 151 that are configured to generate and detect radiation rays. In some embodiments, the cavity 151 may accommodate a ray emitting device 154 and a detector 153. The ray emitting device 154 can emit radiation rays. The radiation rays may be emitted at a subject placed in the cavity 151 and transmitted through the subject to be received by the detector 153. The radiation rays may include particulate rays, photon rays, or the like, or a combination thereof. The particulate rays may include neutrons, protons, electrons, mu-medium, heavy ions, or the like, or a combination thereof. Photon rays may include X-rays, γ-rays, α-rays, β-rays, ultraviolet rays, lasers, or the like, or a combination thereof. Merely by way of example, the photon rays may be X-rays, and the corresponding medical scanning device 150 thereof may be one or more of a CT system, a digital radiography (DR) system, a multimodal medical imaging system, and the like. Further, in some embodiments, a multimodal medical imaging system may include one or more of a positron emission tomography-computed tomography-(PETCT) system, a SPECT/MRI system, or the like. As another example, the ray emitting device 154 may be an X-ray tube. The X-ray tube may emit X-rays that pass through a subject disposed inside the cavity 151 and are received by the detector 153.

The detector 153 may include a plurality of detector pixel units. As used herein, a detector pixel unit refers to the smallest imaging unit on the detector. In some embodiments, the plurality of detector pixel units on the detector 153 may be arranged in a preset manner, e.g., the plurality of detector pixel units are arranged in m rows and n columns, wherein the row may be in a row direction of the detector 153 and the column may be in a channel direction of the detector 153. In some embodiments, the detector 153 may be a circular detector, a square detector, an arcuate detector, etc. A rotation angle of the arcuate detector may be between 0° and 360°. In some embodiments, the rotation angle of the arcuate detector may be fixed. In some embodiments, the rotation angle of the arcuate detector may be adjusted as desired. For example, the rotation angle of the arcuate detector may be adjusted based on a desired resolution of the image, a size of the image, a sensitivity of the detector, a stability of the detector, or a combination thereof. In some embodiments, the detector 153 may be a one-dimensional detector, a two-dimensional detector, or a three-dimensional detector.

The bed frame 152 may support a subject to be detected (e.g., a patient to be tested, a homogeneous plate, etc.).

The operation control computer device may be associated with the cavity 151, the ray emitting device 154, the detector 153, a high voltage generator, the bed frame 152, and/or the image generator. The aforementioned devices may be connected to each other in direct or indirect manners. In some embodiments, the operation control computer device may control the cavity 151 to rotate to a certain position. The position may be a system default or may be set by a user (e.g., a doctor, a nurse, etc.). In some embodiments, the operation control computer device may control the high voltage generator. For example, the operation control computer device may control the intensity of voltage or current generated by a high voltage generator.

The image generator may generate an image. In some embodiments, the image generator may perform operations such as image preprocessing, image reconstruction, and/or region of interest extraction to generate a medical image of the user. The image generator may be associated with the detector 153, the operation control computer device, and/or an external data source (not illustrated in the figure). In some embodiments, the image generator may receive data from the detector 153 or the external data source and generate the medical image of the user based on the received data. The external data source may be one or more of a hard disk, a floppy disk, a random access memory (RAM), a dynamic random access memory (DRAM), or the like.

It should be noted that the description of the application scenario 100 may be merely provided for the purpose of illustrating and is not intended to limit the scope of the present disclosure. For those skilled in the art, a variety of amendments or variations may be made based on the description in the present disclosure. For example, the application scenario 100 may also include a database. However, these amendments or variations may not depart from the scope of the present disclosure.

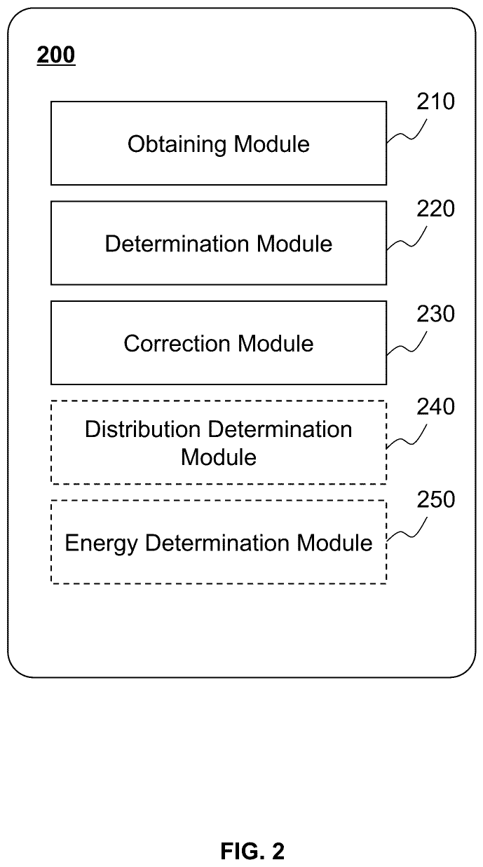

FIG. 2 is a block diagram illustrating a system 200 for data correction according to some embodiments of the present disclosure.

As shown in FIG. 2, the system 200 for data correction may include an obtaining module 210, a determination module 220, and a correction module 230.

The obtaining module 210 may be configured to obtain spatial positions of the detector pixel units and determine cosine correction data based on the spatial positions of the detector pixel units and the focal point of the ray emitting device. In some embodiments, the obtaining module 210 may be configured to obtain three-dimensional coordinates of the detector pixel units and determine the cosine correction data based on the spatial positions of the detector pixel units and coordinates of the focal point of the ray emitting device. In some embodiments, the obtaining module 210 may also be configured to: obtain a projection image of the testing phantom on the detector; determine a response center point of the detector based on the projection image; and determine the three-dimensional coordinates of the detector pixel units based on the response center point, the distances between the plurality of detector pixel units, and the distance between the focal point of the ray emitting device and the response center point. In some embodiments, the obtaining module 210 may further be configured to: for each of the detector pixel units, determine the cosine of an angle between a first connecting line and a second connecting line based on the three-dimensional coordinates of the detector pixel units and the coordinates of the focal point to obtain the cosine correction data, wherein the first connecting line refers to a connecting line between the detector pixel unit and the focal point of the ray emitting device, the second connecting line refers to a connecting line between the focal point of the ray emitting device and a vertical point on the detector, and the second connecting line is perpendicular to the detector.

The determination module 220 may be configured to determine response data to be corrected of the detector and correct the response data to be corrected using the cosine correction data to obtain target data. In some embodiments, the determination module 220 may further be used for obtaining, via the detector, phantom data and air data, wherein the phantom data may be response data of the detector when a testing phantom is disposed between the ray emitting device and the detector, and the air data may be the response data of the detector when a testing phantom is not disposed between the ray emitting device and the detector, and determining the response data to be corrected based on the air data and the phantom data.

The correction module 230 may be configured to determine one or more correction coefficients corresponding to the detector pixel units based on the target data and correct the response data of the subject to be detected based on the one or more correction coefficients. In some embodiments, the correction module 230 may also be configured to: for each row of detector pixel units of the detector in a preset direction, determine a mean value of the target data corresponding to the row of the detector pixel units, designate the mean value as the correction data of the row of the detector pixel units; and determine the one or more correction coefficients based on the correction data of all the detector pixel units.

In some embodiments, the response data to be corrected may include data of the detector in response to homogeneous plates of different thicknesses. In some embodiments, the correction module 230 may further be configured to: establish a linear relationship between the target data and the correction data of the all the plurality of detector pixel units; and determine, based on the correction data of all the detector pixel units corresponding to the homogeneous plates of the different thicknesses, one or more correction coefficients by solving the linear relationship between the target data and the correction data of the all the plurality of detector pixel units.

In some embodiments, the data correction system 200 may further include a distribution determination module 240, which may be configured to: determine a plurality of homogeneous plate combinations, the homogeneous plate combinations including homogeneous plates of at least two thicknesses, the homogeneous plates of at least two thicknesses may include at least two base substances; obtain air correction data for the detector response to each of the plurality of homogeneous plate combinations; determine decomposition coefficients corresponding to different thicknesses of homogeneous plates for each material based on the air correction data for the detector response to each homogeneous plate combination; determine a distribution of base substances in the subject to be detected based on the response data of the subject to be detected and the decomposition coefficients.

In some embodiments, the data correction system 200 may further include an energy determination module 250. The energy determination module 250 may be configured to determine an effective attenuation coefficient of the homogeneous plate based on the correction data in response to homogeneous plates of different thicknesses and determine an effective energy of the ray emitting device or detector based on the effective attenuation coefficient of the homogeneous plate.

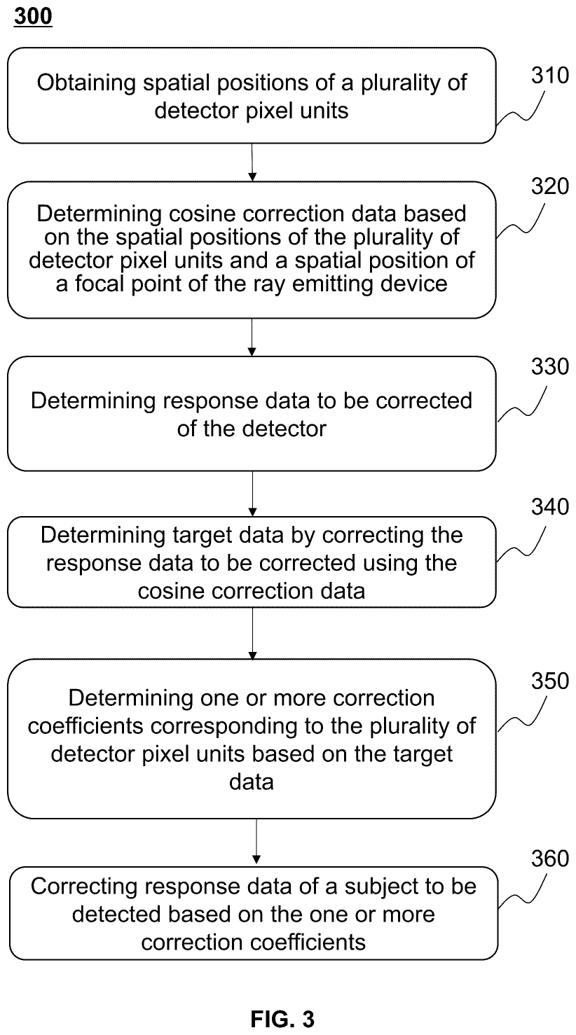

More description of the obtaining module 210, the determination module 220, the correction module 230, the distribution determination module 240, and the energy determination module 250 may be found in FIG. 3 and its related descriptions, and will not be repeated here.

It should be noted that the above description of the data correction system 200 and its modules may be merely provided for the purpose of description convenience and does not limit the present disclosure to the scope of the cited embodiments. It should be understood that for those skilled in the art, after understanding the principle of the system, it may be possible to make any combination of the individual modules or form a sub-system to connect with other modules without departing from this principle. In some embodiments, the obtaining module 210, the determination module 220, the correction module 230, the distribution determination module 240, and the energy determination module 250 disclosed in FIG. 2 may be different modules in a single system, or a single module that can fulfill the functions of two or more of the aforementioned modules. For example, the individual modules may share a common storage module, and the individual modules may each have their own storage module. Variations such as these are within the scope of protection of the present disclosure.

FIG. 3 is a flowchart illustrating an exemplary process 300 for data correction according to some embodiments of the present disclosure. As shown in FIG. 3, the process 300 may include the steps described below. In some embodiments, the process 300 may be applied to a medical scanning device. The medical scanning device may include a ray emitting device and a detector, and more descriptions of the medical scanning device may be found in FIG. 1 and the related descriptions thereof, which will not be repeated herein. In some embodiments, the process 300 may be performed by the processor 110 or the data correction system 200.

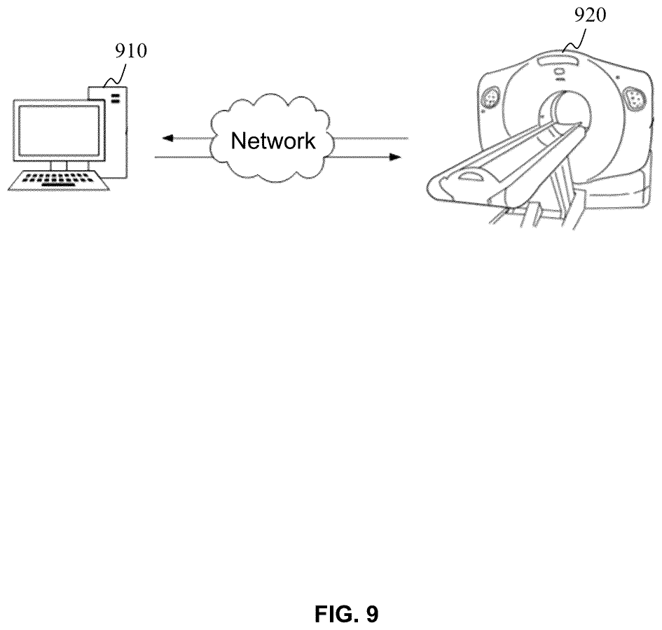

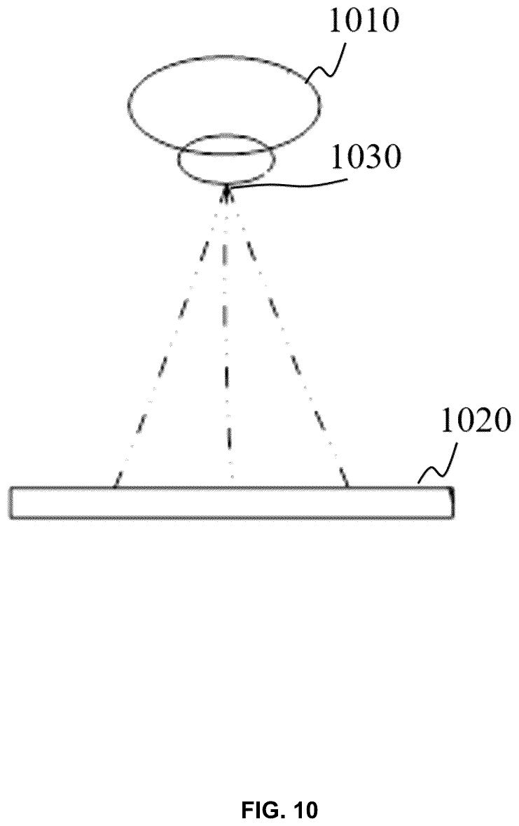

The method for data correction provided by embodiments of the present disclosure may be applied in an application environment including a terminal 910 and a medical scanning device 920, as shown in FIG. 9. The terminal 910 may communicate with the medical scanning device 920 via a network. The terminal 910 may be, but is not limited to, various personal computers, laptop machines, and tablet computers. The medical scanning device 920 may be, but is not limited to, a computed tomography (CT) device and a positron emission computed tomography (PET)-CT device. The structure of the medical scanning device 920 may be illustrated in FIG. 10. The medical scanning device 920 may include a ray emitting device 1010 and a detector 1020. The ray emitting device 1010 may be an X-ray emitting device, such as an X-ray tube. The detector 1020 may include a block of semiconductor crystal material and a plurality of detector pixel units. Each of the plurality of detector pixel units may include an application-specific integrated circuit (ASIC), and the ASIC may include a charge-sensitive preamplifier, a pulse rectifier, a comparator, and a digital counter. The ray emitting device 1010 may be configured to emit radiation rays through a focal point 1030 of the ray emitting device. The radiation rays may pass through a subject to be scanned in the medical scanning device 920 and may form a projection of the subject to be scanned on the detector 1020. The embodiments of the present disclosure do not limit the types and structures, etc., of the ray emitting device 1010 and the detector 1020, as long as their functions are realized.

In 310, spatial positions of a plurality of detector pixel units are obtained. In some embodiments, the operation 310 may be performed by the obtaining module 210.

A spatial position of a detector pixel unit may characterize location information of the detector pixel unit in a three-dimensional space. The spatial position of the detector pixel unit may be expressed in various forms. For example, the spatial position of the detector pixel unit may be expressed as a relative position relationship to a reference point (e.g., a center of the detector). As another example, the spatial position of the detector pixel unit may be expressed by a three-dimensional coordinate system. Exemplarily, the spatial position of the detector pixel unit may be three-dimensional Cartesian coordinates of the detector pixel unit in a three-dimensional Cartesian coordinate system. As another example, the spatial position of the detector pixel unit may be cylindrical coordinates of the detector pixel unit in a cylindrical coordinate system. In some embodiments, the obtaining module 210 may obtain the three-dimensional coordinates of the plurality of detector pixel units in any feasible manner. For example, the three-dimensional coordinates of the plurality of detector pixel units may be obtained directly from the user terminal 130, the storage device 140, the medical scanning device 150, or an external data source.

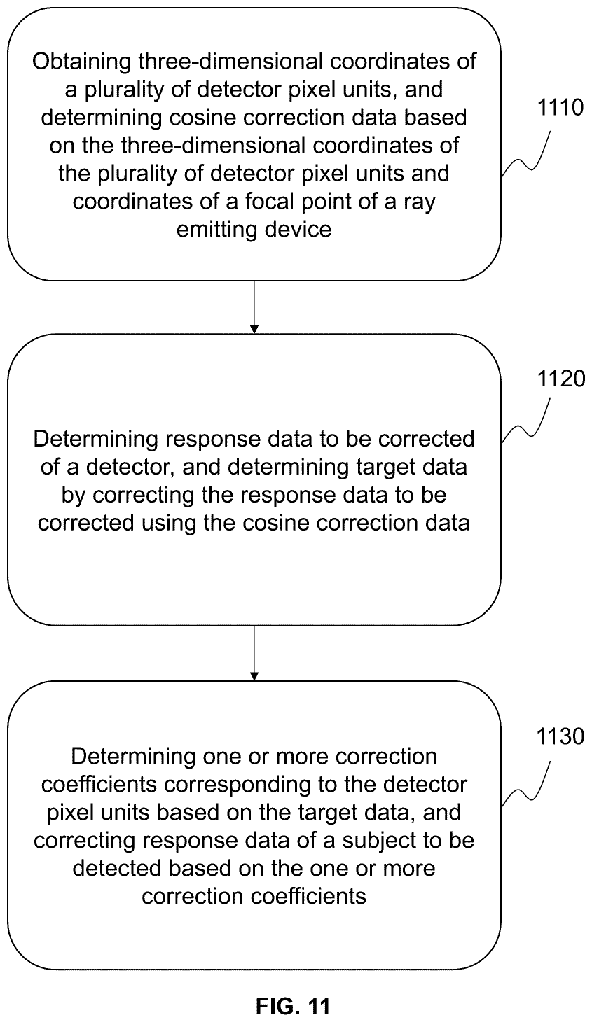

In some embodiments, as shown in FIG. 11, a method 1100 for data correction is provided, taking the method 1100 applied to the terminal 910 in FIG. 9 as an example, the method 1100 may include operation 1110, i.e., obtaining three-dimensional coordinates of the plurality of detector pixel units.

In some embodiments, the obtaining module 210 may also determine the three-dimensional coordinates of the plurality of detector pixel units through a testing phantom. For example, the obtaining module 210 may obtain a projection image of the testing phantom on the detector, determine a response center point of the detector based on the projection image, determine the three-dimensional coordinates of the plurality of detector pixel units based on the response center point, distances between the plurality of detector pixel units, and a distance between the focal point of the ray emitting device and the response center point.

The testing phantom may be an object that is scanned by the medical scanning device 150 to obtain the three-dimensional coordinates of the plurality of detector pixel units. For example, the testing phantom may include homogeneous plates, steel balls, or the like.

Three-dimensional coordinates of a detector pixel unit refer to three-dimensional coordinates of the detector pixel unit on the detector determined with a rotation center as a coordinate origin. The coordinates of the focal point of the ray emitting device refer to the three-dimensional coordinates of the focal point determined with the rotation center as the coordinate origin. The rotation center refers to a center of the ray emitting device and the detector while rotating in a circle around the subject to be detected, and the rotation center may be a virtual point. Specifically, when the medical scanning device is a CT device, the rotation center may refer to a point on a centerline of an aperture of the CT device.

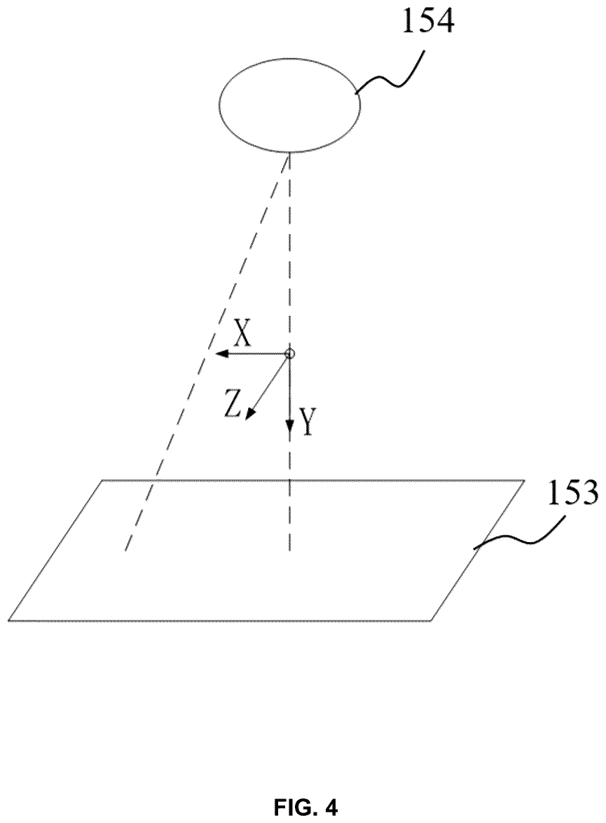

FIG. 4 is a schematic diagram illustrating a three-dimensional Cartesian coordinate system in which three-dimensional coordinates of detector pixel units are located according to some embodiments of the present disclosure. As shown in FIG. 4, in some embodiments, a three-dimensional Cartesian coordinate system may be established with the rotation center of the ray emitting device as the coordinate origin, a theoretical center projection line of the ray emitting device as the Y-axis, a row direction of the detector as the X-axis, and the channel direction of the detector as the Z-axis. The three-dimensional coordinates of the plurality of detector pixel units may be the coordinates of the plurality of detector pixel units in the three-dimensional Cartesian coordinate system. The theoretical center projection line of the ray emitting device refers to a projection line of the focal point of the ray emitting device projected vertically to the detector.

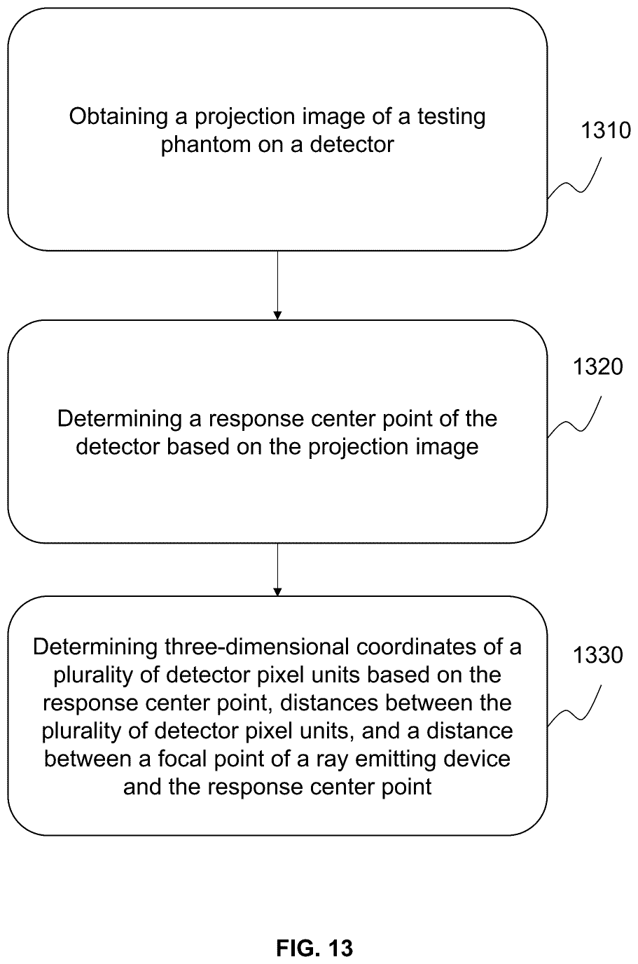

In an embodiment, as shown in FIG. 13, a process for obtaining the three-dimensional coordinates of the plurality of detector pixel units may include the following operations.

In 1310, a projection image of a testing phantom on the detector is obtained.

The ray emitting device and the detector are rotated around the testing phantom. Through the rotation of the ray emitting device and the detector, projection images of the testing phantom on the detector at different angles can be obtained. Specifically, the projection images may be transmitted to the terminal after being obtained by the detector and stored in a memory of the terminal, and the terminal may obtain the projection images directly in the memory when needed. The testing phantom may be a homogeneous plate or a steel ball phantom. The present embodiment does not limit the type and structure, etc., of the testing phantom, as long as the function of the testing phantom is realized.

In 1320, a response center point of the detector is determined based on the projection image.

After obtaining a plurality of projection images of the testing phantom on the detector, the terminal may determine a response center point of the detector based on the projection images. Specifically, the terminal may analyze each of the obtained projection images, determine a center of mass of each of the projection images, connect the centers of mass of all the projection images, and solve a center point of an image obtained after all the centers of mass are connected. The center point is the response center point of the detector.

In 1330, the three-dimensional coordinates of the plurality of detector pixel units are determined based on the response center point, distances between the plurality of detector pixel units, and a distance between the focal point of the ray emitting device and the response center point.

The distances between the plurality of detector pixel units may be pre-stored by the staff in the memory of the terminal, and the terminal may obtain the distances directly in the memory when needed. After obtaining the response center point of the detector, the terminal may determine the three-dimensional coordinates of the plurality of detector pixel units based on the response center point, the distances between the plurality of detector pixel units, and the distance between the focal point of the ray emitting device and the response center point.

Specifically, the three-dimensional coordinates of the plurality of detector pixel units may be determined with the rotation center of the ray emitting device and the detector when rotating around the subject to be detected as the coordinate origin. The response center point, the rotation center, and the focal point of the ray emitting device may be in the same straight line. The three-dimensional coordinates of the response center point may be obtained based on the coordinates of the focal point of the ray emitting device and the distance between the focal point of the ray emitting device and the response center point. According to the three-dimensional coordinates of the response center point, a distance between the response center point and its nearest detector pixel unit, the three-dimensional coordinates of the detector pixel unit may be determined. According to the distances between the plurality of detector pixel units, the three-dimensional coordinates of the other detector pixel units may be determined.

The method for determining three-dimensional coordinates of the detector pixel units provided in this embodiment may be simple to understand and easy to implement.

The distances between the plurality of detector pixel units may be distances between every two adjacent detector pixel units. In some embodiments, the obtaining module 210 may obtain the distances between the plurality of detector pixel units in any feasible manner. For example, the distances between the plurality of detector pixel units may be obtained directly from the user terminal 130, the storage device 140, the medical scanning device 150, or the external data source.

In some embodiments, the obtaining module 210 may obtain a distance between the focal point of the ray emitting device and the rotation center, determine the three-dimensional coordinates (0,−y_{tube},0) of the focal point of the ray emitting device, wherein y_{tube} is the distance between the focal point of the ray emitting device and the rotation center.

In some embodiments, the obtaining module 210 may determine the three-dimensional coordinates (0,y_{center},0) of the response center point of the detector based on the three-dimensional coordinates of the focal point of the ray emitting device, wherein y_{center} denotes the distance between the response center point and the rotation center. It should be appreciated that y_{center}=yₜₒₜₐₗ−y_{tube}, wherein yₜₒₜₐₗ denotes the distance between the focal point of the ray emitting device and the response center point.

In some embodiments, the obtaining module 210 may obtain the distance between the focal point of the ray emitting device and the response center point in any feasible manner. For example, the distance between the focal point of the ray emitting device and the response center point may be obtained directly from the user terminal 130, the storage device 140, the medical scanning device 150, or the external data source.

In some embodiments, after obtaining the three-dimensional coordinates of the response center point, the obtaining module 210 may determine the three-dimensional coordinates of each of the plurality of detector pixel units based on the three-dimensional coordinates of the response center point and the distances between the plurality of detector pixel units. For example, the obtaining module 210 may first determine the three-dimensional coordinates of multiple detector pixel units (i.e., first adjacent detectors) adjacent to the response center point, then determine the three-dimensional coordinates of multiple detector pixel units (i.e., second adjacent detectors) adjacent to each of the first adjacent detectors, and so on until the three-dimensional coordinates of all the plurality of detector pixel units are determined. Merely by way of example, when the left side of the response center point is in a positive direction of the X-axis, the three-dimensional coordinates of a detector pixel unit adjacent to the left side of the response center point may be (x_{space},y_{center},0), wherein x_{space} denotes the distance between the response center point and the detector pixel unit adjacent to the left side of the response center point; and the three-dimensional coordinates of the detector pixel units adjacent to the right side of the response center point may be (−x_{space},y_{center},0).

In some embodiments, three-dimensional coordinates of the plurality of detector pixel units may be quickly and accurately determined based on the response center point, the distances between the plurality of detector pixel units, and the distance between the focal point of the ray emitting device and the response center point.

In 320, cosine correction data is determined based on the spatial positions of the plurality of detector pixel units and a spatial position of the focal point of the ray emitting device. In some embodiments, the operation 320 may be performed by the obtaining module 210.

The spatial position of the focal point of the ray emitting device may be configured to characterize the spatial position of the focal point of the ray emitting device. The spatial position of the focal point of the ray emitting device may be represented in any form. For example, the spatial position of the focal point of the ray emitting device may be represented as a relative position relationship to a reference point (e.g., the center of the detector). As another example, the spatial positions of the detector pixel units may be represented by three-dimensional coordinates (i.e., the coordinates of the focal point).

The cosine correction data may be configured to characterize an angle between a connecting line connecting each of the plurality of detector pixel units and the focal point of the ray emitting device and a theoretical center projection line of the ray emitting device.

In some embodiments, as shown in FIG. 11, operation 1110 may further include determining the cosine correction data of the plurality of detector pixel units based on the three-dimensional coordinates of the plurality of detector pixel units and the coordinates of the focal point of the ray emitting device.

The detector may include a plurality of detector pixel units. The terminal may obtain three-dimensional coordinates of each of the plurality of detector pixel units and the coordinates of the focal point of the ray emitting device, and determine the cosine correction data between the three-dimensional coordinates of each of the plurality of detector pixel units and the coordinates of the focal point of the ray emitting device based on the three-dimensional coordinates of each of the plurality of detector pixel units and the coordinates of the focal point of the ray emitting device. For each of the plurality of detector pixel units of the detector, a piece of cosine correction data may be determined, so for the plurality of detector pixel units on the detector, a plurality of pieces of cosine correction data may be obtained. The embodiment does not limit the specific process of determining the cosine correction data, as long as the function can be realized.

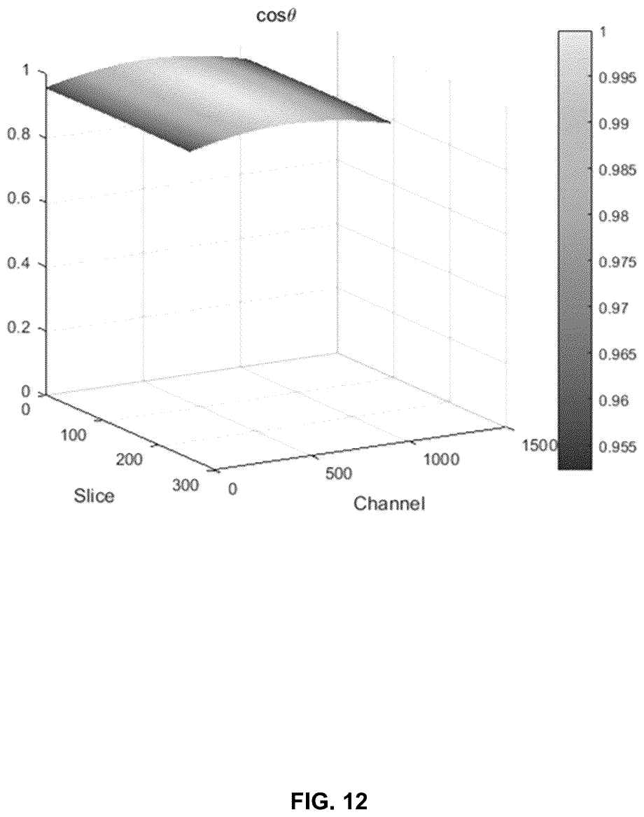

In an optional embodiment, the plurality of detector pixel units on the detector may be arranged in a preset manner, e.g., the plurality of detector pixel units may be arranged in m rows and n columns, wherein the row is defined in terms of a row direction of the detector and the column is defined in terms of a channel direction of the detector. As shown in FIG. 4, the row direction of the detector may be a Z-axis direction in the three-dimensional coordinate system where the detector pixel units are located, and the channel direction of the detector may be an X-axis direction in the three-dimensional coordinate system where the detector pixel units are located. Specifically, the plurality pieces of cosine correction data may be represented using a matrix in the form of S_{i,j}, i=1, 2 . . . m, j=1, 2 . . . n. The rows and columns of the matrix correspond to the rows and columns of the detector pixel units, for example, the element s_{i,j} of the i^{th} row and j^{th} column of a first matrix S may characterize an angle cos θ_{i,j} corresponding to the detector pixel unit of the i^{th} row and j^{th} column of the detector. The matrix of the cosine correction data is shown in FIG. 12, wherein Channel denotes the channel direction of the detector and Slice denotes the row direction of the detector.

In some embodiments, for each detector pixel unit, the obtaining module 210 may determine an angle corresponding to the detector pixel unit through the three-dimensional coordinates of the detector pixel unit and the coordinates of the focal point of the ray emitting device. Then the obtaining module 210 may determine the cosine correction data by combining the angles corresponding to the plurality of detector pixel units according to the arrangement manner of the plurality of detector pixel units on the detector.

In some embodiments, for each of the plurality of detector pixel units, the cosine correction data may be obtained by determining the cosine of an angle between a first connecting line and a second connecting line based on the three-dimensional coordinates of the detector pixel unit and the coordinates of the focal point. The first connecting line is a connecting line between the detector pixel unit and the focal point of the ray emitting device, the second connecting line is a connecting line between the focal point of the ray emitting device and a vertical point on the detector, and the second connecting line may be perpendicular to the detector. The obtaining module 210 may determine the length of the first connecting line based on the three-dimensional coordinates of the detector pixel unit and the coordinates of the focal point, and then determine the cosine of the angle of the detector pixel unit in combination with a length of the second connecting line, thereby determining an angle corresponding to the detector pixel unit. It should be understood that a ratio of the length of the second connecting line to the length of the first connecting line may be the cosine of the angle of the detector pixel unit.

Both the first connecting line and the second connecting line are virtual connecting lines and do not actually exist. The vertical point may also be a virtual point, and the vertical point is an intersection of the second connecting line with the detector. For one of the plurality of detector pixel units, when determining the cosine correction data, the terminal may first determine an angle between the first connecting line (a connecting line between the detector pixel unit and the focal point of the ray emitting device) and the second connecting line (a connecting line between the focal point of the ray emitting device and the vertical point on the detector), and determine a value of cosine of the angle, i.e., a ratio of a distance between the focal point of the ray emitting device and the detector pixel unit to a distance between the focal point of the ray emitting device and the vertical point on the detector, such that obtain the cosine correction data of the detector pixel unit. The cosine correction data of all the plurality of detector pixel units in the detector may be obtained through the same process.

The three-dimensional coordinates of one of the plurality of detector pixel units may be expressed as (x_{i,j},y_{i,j},z_{i,j}), the three-dimensional coordinates of the focal point of the ray emitting device may be expressed as (0,−y_{tube},0), then a distance l_{i,j} between the focal point of the ray emitting device and the detector pixel unit may be expressed as

$$\\ {l}_{i , j} = \sqrt{{x}_{i , j}^{{}_{} 2} + {( {y}_{i , j} - {y}_{tube} )}^{2} + {z}_{i , j}^{{}_{} 2}} .$$

The distance between the focal point of the ray emitting device and the vertical point on the detector may be expressed as k_{i,j}, then the cosine correction data may be expressed as cos θ_{i,j}=kᵢⱼ/l_{i,j}.

The present embodiment provides a simple and fast method for determining cosine correction data, which may improve the efficiency of determining the one or more correction coefficients.

In some embodiments, the vertical point may be the response center point described above. After determining the response center point, the second connecting line may be determined directly from the response center point and the focal point of the ray emitting device without re-determining the vertical point, which may improve the efficiency of determining the cosine correction data.

In some embodiments, according to the three-dimensional coordinates of the detector pixel unit and the coordinates of the focal point, the cosine correction data may be determined accurately and quickly by determining the cosine of the angle between the first connecting line and the second connecting line, thereby quickly determining the angle between the connecting line connecting the detector pixel unit and the focal point of the ray emitting device and the theoretical center projection line of the ray emitting device, which can facilitate the determining the one or more correction coefficients of the detector pixel units.

In 330, response data to be corrected of the detector is determined. In some embodiments, the operation 330 may be performed by the determination module 220.

The response data to be corrected may be data related to the response data of each of the plurality of detector pixel units in the detector after being scanned by the ray emitting device. For example, the response data to be corrected refers to the response data of each of the plurality of detector pixel units in the detector after rays emitted by the ray emitting device are emitted to the detector through the testing phantom. The type of the testing phantom, the structure of the testing phantom, and the material of the testing phantom may be known. When the response data to be corrected is needed to obtain, the determination module 220 may control the medical scanning device 150 to perform at least one scan to obtain the response data to be corrected.

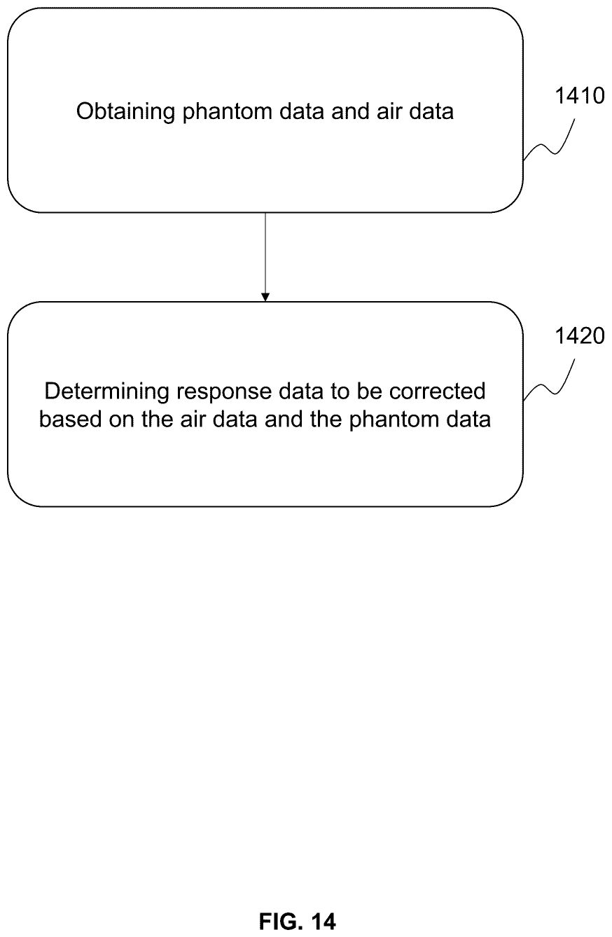

In an embodiment, as shown in FIG. 14, a possible process for obtaining the response data to be corrected of the detector may include the following operations.

In 1410, phantom data and air data is obtained. The phantom data may be response data of the detector when a testing phantom is disposed between the ray emitting device and the detector, and the air data may be response data of the detector when the testing phantom is not disposed between the ray emitting device and the detector.

The testing phantom may include homogeneous plates of different thicknesses, e.g., a polymethylmethacrylate plate or an aluminum plate. The testing phantom is disposed between the ray emitting device and the detector, and the rays emitted by the ray emitting device are transmitted to the detector after passing through the testing phantom, and the response data of each of the plurality of detector pixel units in the detector may be recorded as the phantom data. The response data of the detector when the testing phantom is not disposed between the ray emitting device and the detector, that is, the rays emitted by the ray emitting device may be transmitted to the detector through the air, and the response data of each detector pixel unit in the detector may be recorded as the air data. The phantom data of the testing phantom of different thicknesses may be expressed as C_{i,j}^{t}, i=1, 2 . . . m, j=1, 2 . . . n, wherein t denotes the thickness of the testing phantom. The air data may be expressed as C_{i,j}^{air}, i=1, 2 . . . m, j=1, 2 . . . n.

In some embodiments, the phantom may include homogeneous plates of different thicknesses of the same material, homogeneous plates of the same thickness of different materials, homogeneous plates of different thicknesses of different materials, or any combination thereof. For example, the phantom may include polymethylmethacrylate plates with thicknesses of 10 mm, 8 mm, and 6 mm. As another example, the phantom may include a 10 mm thick polymethylmethacrylate plate and a 10 mm thick aluminum plate. As another example, the phantom may include a 10 mm thick polymethylmethacrylate plate, a 6 mm thick polymethylmethacrylate plate, and an 8 mm thick aluminum plate.

In 1420, the response data to be corrected is determined based on the air data and the phantom data.

After obtaining the air data and the phantom data, the terminal may determine a ratio of the air data to the phantom data and calculate the logarithm of the ratio, thus the response data to be corrected may be obtained. Specifically, the response data to be corrected M_{i,j} may be expressed as M_{i,j}=ln(C_{i,j}^{air}/C_{i,j}^{t}).

In this embodiment, the effect of air on the rays emitted by the ray emitting device is considered when determining the response data to be corrected, so that the determined response data to be corrected can be closer to an actual application scenario, thus the correction coefficients determined based on this response data to be corrected can be more accurate, and a correction effect of the inhomogeneity of the response of the detector can be improved. Moreover, when the testing phantom is a homogeneous plate, the influence of differences in the lengths of the rays passing through the homogeneous plate from different directions on the finalized correction coefficients may be eliminated, thereby causing the determined correction coefficients to be more accurate.

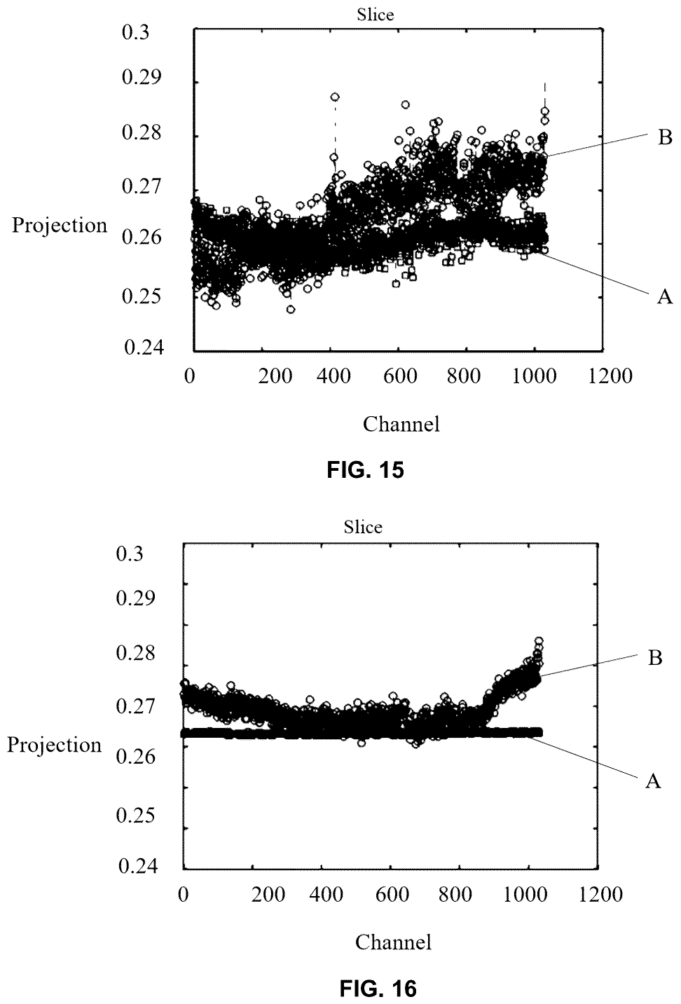

In a specific embodiment, when the testing phantom is a 10 mm thick polymethylmethacrylate plate, the corresponding response data to be corrected before correction is shown in FIG. 15. When the testing phantom is a 100 mm thick polymethylmethacrylate plate, the corresponding response data to be corrected after correction is shown in FIG. 16. The horizontal coordinate in FIG. 15 and FIG. 16 may be the detector pixel units, and the vertical coordinate may be the target data after correcting the response data to be corrected, wherein A indicates the target data after correction and B indicates the response data to be corrected before correction. As can be seen from FIG. 15 and FIG. 16, the fluctuation of the corrected target data may be reduced and the uniformity may be greatly improved.

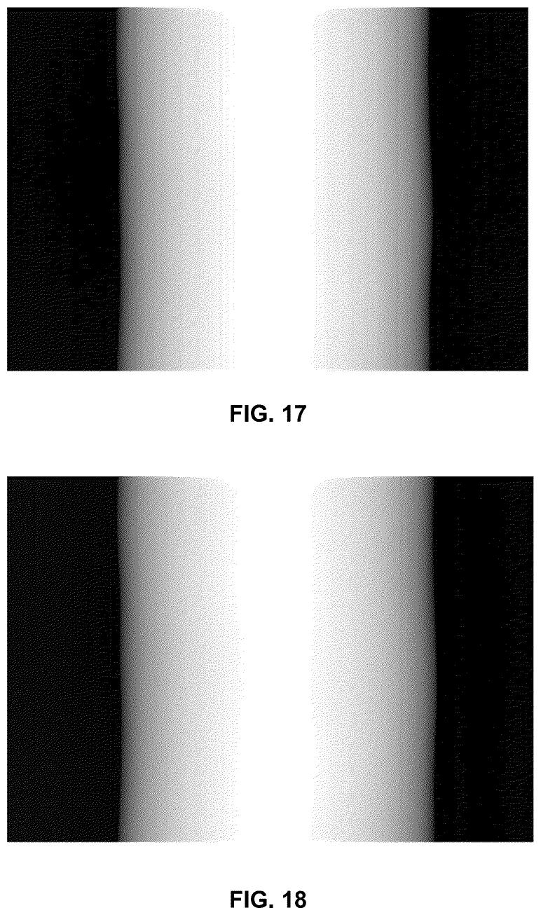

In a specific embodiment, when the subject to be detected is an actual water phantom, a sine diagram before correcting the response data of the actual water phantom may be shown in FIG. 17, and the sine diagram after correcting the response data of the actual water phantom using the method for data correction provided in the present disclosure may be shown in FIG. 18. By comparing FIG. 17 and FIG. 18, it can be seen that the uniformity of the sine diagram after correction may be improved and the barring artifacts may be weakened.

In some embodiments, the response data to be corrected corresponding to each homogeneous plate may be represented by a fourth matrix, wherein an element of the fourth matrix may characterize an air correction value of one detector pixel unit corresponding to the homogeneous plate. For example, an element of the i^{th} row and the j^{th} column of the fourth matrix m_{i,j}=ln(c_{i,j}^{air}/c_{i,j}^{t}) may characterize the data corresponding to the i^{th} row and the j^{th} column of the detector pixel units in the detector.

In 340, the processor may obtain target data by correcting the response data to be corrected using the cosine correction data. In some embodiments, the operation 340 may be performed by determination module 220.

The target data may be data after the response data to be corrected eliminates differences in the lengths of the rays passing through the homogeneous plates from different directions. In some embodiments, the determination module 220 may correct the response data to be corrected using the cosine correction data to obtain the target data. For the air correction data corresponding to each homogeneous plate, the processor may correct the air correction data using the angle corresponding to each detector pixel unit to obtain the target data corresponding to each homogeneous plate.

In some embodiments, a method for data correction as shown in FIG. 11 may further include operation 1120. In 1120, the response data to be corrected of the detector may be determined and the target data may be obtained by correcting the response data to be corrected of the detector using the cosine correction data. After obtaining the response data to be corrected of the detector, the terminal may correct the response data to be corrected using the determined cosine correction data to obtain the corrected data, i.e., the target data. The present embodiment does not limit the method for determining the response data to be corrected of the detector, as long as the function can be realized.

Specifically, the response data to be corrected of the detector may be represented as a matrix, which may be represented as M_{i,j}, i=1, 2 . . . m, j=1, 2 . . . n. The terminal may multiply the cosine correction data (in the form of a matrix) with the response data to be corrected (by multiplying the elements at corresponding positions in the matrix) to obtain the target data, and the target data B_{i,j} may be expressed as: B_{i,j}=S_{i,j}⊗M_{i,j}, wherein Ð denotes a Hadamard product, i.e., an operation of multiplying the elements of corresponding positions of two matrices.

In some embodiments, the determination module 220 may correct the response data to be corrected using the cosine correction data, which may eliminate the influence of differences in the lengths of the rays passing through the homogeneous plate from different directions on the finalized correction coefficients, thereby causing the determined correction coefficients to be more accurate.

In 350, the one or more correction coefficients corresponding to the plurality of detector pixel units may be determined based on the target data. In some embodiments, the operation 350 may be performed by the correction module 230.

The correction coefficient(s) may be parameters configured to correct the response data of the plurality of detector pixel units to reduce the artifacts.

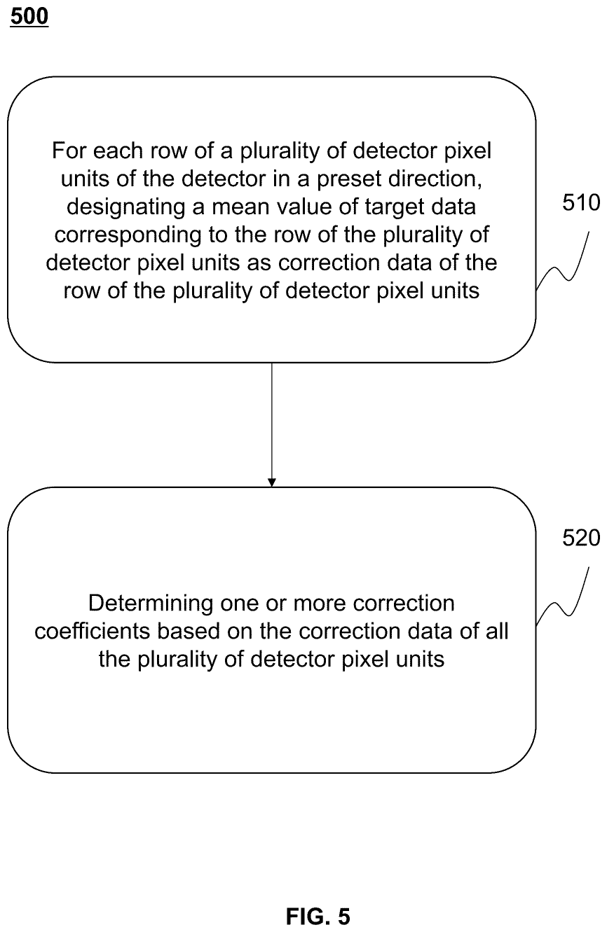

In some embodiments, the correction module 230 may determine the correction coefficient(s) based on a mean value of the target data corresponding to each row of the plurality of detector pixel units. The determination of the correction coefficient(s) based on the mean value of the target data corresponding to each row of the plurality of detector pixel units may be found in FIG. 5 and the related descriptions thereof, and will not be repeated herein.

In 360, the response data of the subject to be detected is corrected based on the one or more correction coefficients. In some embodiments, the operation 360 may be performed by the correction module 230.

In some embodiments, the correction module 230 may first determine the response data of the subject to be detected. The subject to be detected may be placed between the ray emitting device and the detector, and the medical scanning device 150 may scan the subject to be detected to obtain the scanning data of the subject to be detected. The scanning data of the subject to be detected may be expressed by a fifth matrix D_{i,j}, wherein an element of the fifth matrix may characterize the response data of a detector pixel unit in response to the subject to be detected. For example, an element d_{i,j} in the i^{th} row and the j^{th} column of the fifth matrix D_{i,j} may characterize the response data of the subject to be corrected corresponding to the detector pixel unit of the i^{th} row and the j^{th} column of the detector.

In some embodiments, the correction module 230 may determine the air correction data

$$\\ ln ( \frac{{C}_{i , j}^{{}_{} air}}{{D}_{i , j}} )$$

of the subject to be detected based on the air data and the scanning data of the subject to be detected. The processor may correct the air correction data ln(C_{i,j}^{air}/D_{i,j}) of the subject to be detected using the one or more correction coefficients to obtain the corrected data.

The method for data correction as shown in FIG. 11 may further include an operation 1130. In 1130, the one or more correction coefficients corresponding to the plurality of detector pixel units are determined based on the target data, and the response data of the subject to be detected is corrected based on the one or more correction coefficients.

The response data of the subject to be detected refers to response data of each of the plurality of detector pixel units in the detector after the rays emitted by the ray emitting device passing through the subject to be detected and being emitted to the detector. After obtaining the target data, the terminal may determine the correction coefficient(s) for correcting the detector pixel unit(s) based on the target data. The response data of the subject may be corrected using the correction coefficient(s). The present embodiment does not limit the specific method of determining the one or more correction coefficients, as long as the function can be realized.

In some embodiments, the method for data correction may include obtaining spatial positions of the plurality of detector pixel units; determining the cosine correction data based on the spatial positions of the plurality of detector pixel units and the spatial position of the focal point of the ray emitting device; determining the response data to be corrected of the detector; determining the target data by correcting the response data to be corrected using the cosine correction data; determining the correction coefficients corresponding to the plurality of detector pixel units based on the target data; and correcting response data of a subject to be detected based on the correction coefficients. According to the method for data correction provided in the embodiment, through the target data obtained by correcting the response data to be detected based on the determined cosine correction data, the correction coefficient(s) for correcting the inhomogeneity of the detector pixel units may be obtained. According to the correction coefficient(s), a more accurate correction of the response data of the subject to be detected may be implemented, such that the effect of correcting the inhomogeneity of the detector pixel units may be improved, thereby enabling a reconstruction image of the subject to be detected to have lower noise and fewer artifacts.

It should be noted that the foregoing description of process 300 is merely provided for the purpose of example and illustration only and is not intended to limit the scope of application of the present disclosure. For those skilled in the art, various amendments and variations may be made to the process 300 under the guidance of the present disclosure. However, these amendments and variations remain within the scope of the present disclosure.

FIG. 5 is a flowchart illustrating an exemplary process for determining a correction coefficient based on a mean value of target data corresponding to each row of the plurality of detector pixel units according to some embodiments of the present disclosure. As shown in FIG. 5, process 500 may include operations described below. In some embodiments, process 500 may be performed by the processor 110.

In 510, for each row of the plurality of detector pixel units of the detector in a preset direction, the processor may determine a mean value of target data corresponding to the row of the plurality of detector pixel units and designate the mean value as correction data of row of the plurality of detector pixel units.

The correction data refers to the data configured to determine the one or more correction coefficients.

The preset direction of the detector may be the row direction of the detector or the channel direction of the detector. If the preset direction is the row direction, the terminal may determine, for each row of the detector pixel units of the detector in the row direction, a mean value of the target data corresponding to the row of the plurality of detector pixel units, and designate the mean value as the correction data of the row of the plurality of detector pixel units. Specifically, the correction data B̅ᵢ corresponding to the i^{th} row of the detector pixel units may be expressed as B̅ᵢ=mean(B_{i,j}).

In some embodiments, for each row of the detector pixel units, the processor may determine the mean value of the target data corresponding to the row of the detector pixel units, thereby determining the correction data of each detector pixel unit in the row of the detector pixel units. For example, for the a^{th} raw of the detector pixel units including n detector pixel units (i.e., a first detector pixel unit, a second detector pixel unit, . . . , and an n^{th} detector pixel unit, etc.), the processor may determine a mean value of target data corresponding to the n detector pixel units, and designate the mean value as the correction data of the n detector pixel units.

In 520, the one or more correction coefficients are determined based on the correction data of all the plurality of detector pixel units.

The one or more correction coefficients refer to one or more coefficients corresponding to correction data obtained after correcting the response data of the detector pixel units. Specifically, the terminal may determine the one or more correction coefficients after obtaining the correction data of all the plurality of detector pixel units of the detector. The determination of the one or more correction coefficients may be determined based on a variation relationship of the inhomogeneity of the detector pixel units.

The method for determining the correction coefficient(s) provided by the embodiment is simple to understand, quick to determine, and easy to implement.

In an optional embodiment, assuming that the inhomogeneity of the detector pixel units is linearly varying, by linearly mapping the target data of the detector pixel units to the correction data, B̅ᵢ=g_{i,j}⊗B_{i,j}+b_{i,j} is obtained, wherein B̅ᵢ denotes a mean value of the target data corresponding to the detector pixel units in the i^{th} row, g_{i,j} denotes a first correction coefficient corresponding to the detector pixel unit of the i^{th} row and the j^{th} column, and b_{i,j} denotes a second correction coefficient corresponding to the detector pixel unit of the i^{th} row and the j^{th} column.

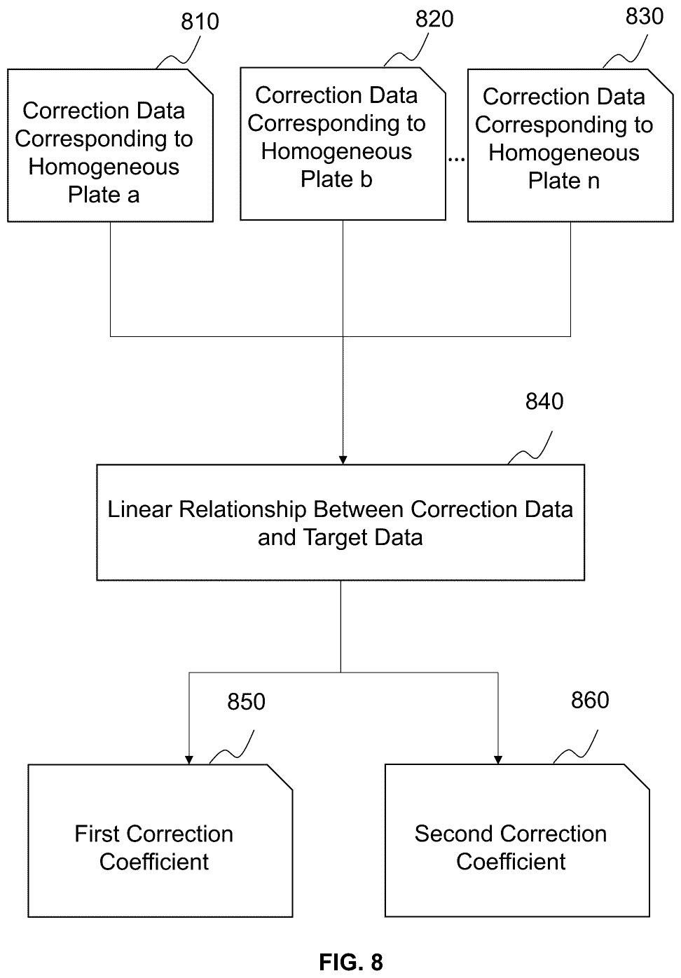

In some embodiments, the processor may determine the one or more correction coefficients by solving the linear relationship between the target data and the correction data of the all the plurality of detector pixel units based on the correction data of all the plurality of detector pixel units corresponding to homogeneous plates of different thicknesses. FIG. 8 is a flowchart illustrating an exemplary process for determining one or more correction coefficients based on correction data of all the plurality of detector pixel units according to some embodiments of the present disclosure. As shown in FIG. 8, for each detector pixel unit, the processor may obtain a first correction coefficient 850 and a second correction coefficient 860 corresponding to the detector pixel unit by solving the linear relationship 840 according to the correction data of the detector pixel unit in response to the homogeneous plates of at least two different thicknesses (e.g., correction data 810 in response to a homogeneous plate a, correction data 820 in response to a homogeneous plate b, and correction data 830 in response to a homogeneous plate n, etc.).

In some embodiments, the processor may correct air correction data of the detector pixel units in response to the subject to be detected based on the linear relationship described above to obtain the corrected data. For example, for the detector pixel unit of the i^{th} row and the j^{th} column, the corrected data of the detector pixel unit may be: D̅_{i,j}=g_{i,j}⊗d_{i,j}+b_{i,j}.

In some embodiments, by establishing a linear relationship between the target data and the correction data of the all the plurality of detector pixel units, the correction coefficient(s) corresponding to each detector pixel unit may be quickly determined.

In some embodiments, for each row of the detector pixel units of the detector in a preset direction, the processor may determine a mean value of target data corresponding to the row of the detector pixel units, and may determine one or more correction coefficients based on the mean value, which may further reduce the volatility of the data configured to determine the correction coefficient(s), so that the determined correction coefficients, after being applied to the correction of the response data of the subject to be detected in a preset direction, may facilitate the uniformity of the reconstruction image.

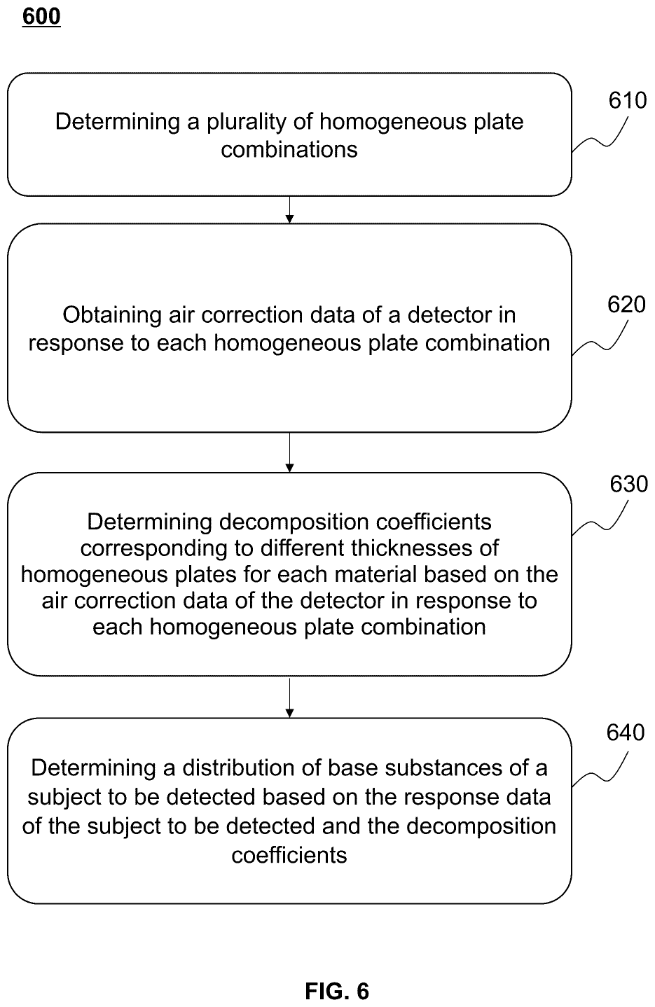

In some embodiments, the method for data correction may also be configured to analyze a distribution of base substances of the subject to be detected. FIG. 6 is a flowchart illustrating an exemplary process for analyzing a distribution of base substances of a subject to be detected according to some embodiments of the present disclosure. As shown in FIG. 6, process 600 may include the following operations.

In 610, a plurality of homogeneous plate combinations may be determined.

A homogeneous plate combination may include homogeneous plates of at least two different thicknesses. For example, a homogeneous plate combination 1 may include a homogeneous plate a with a thickness of tₚ and a homogeneous plate b with a thickness of tₐ. The two different homogeneous plates may be composed of two different base substances. For example, the homogeneous plate a may be a polymethylmethacrylate homogeneous plate and the homogeneous plate b may be a homogeneous aluminum plate.

In 620, air correction data for the detector response to each homogeneous plate combination is obtained.

For each homogeneous plate combination, the ray emitting device may scan the homogeneous plate combination, and the distribution determination module 240 may obtain the air correction data for the detector response to each homogeneous plate of the homogeneous plate combination. More description of obtaining the air correction data of the homogeneous plates may be found in FIG. 3 and its related descriptions, which will not be repeated herein.

In 630, decomposition coefficients corresponding to different thicknesses of homogeneous plates for each material may be determined based on the air correction data for the detector response to each homogeneous plate combination.

In some embodiments, the distribution determination module 240 may determine a relationship among the air correction data of the homogeneous plate combinations, the thickness of each homogeneous plate of the homogeneous plate combination, and the decomposition coefficient corresponding to each base substance, and determine the decomposition coefficient corresponding to each base substance based on the air correction data for the detector response to each homogeneous plate combination and the thickness of each homogeneous plate.

In one embodiment, the method for data correction provided by embodiments of the present disclosure may be configured to analyze the distribution of the base substance composition of the subject to be detected. Specifically, assuming that the testing phantom uses a polymethylmethacrylate homogeneous plate with a thickness of tₚ and a homogeneous aluminum plate with a thickness of tₐ as the base substance, and for a ray emitting device having two energy bins (a high-energy bin H and a low-energy bin L), the thickness of the testing phantom may be expressed by the following equation:

$$\\ {t}_{p} = {m}_{0} + {m}_{1} {B}_{H} + {m}_{2} {B}_{L} + {m}_{3} {B}_{H} {B}_{L} + {m}_{4} {B}_{H}^{{}_{} 2} + {m}_{5} {B}_{L}^{{}_{} 2} + {m}_{6} {B}_{H}^{{}_{} 3} + {m}_{7} {B}_{L}^{{}_{} 3} + {m}_{8} {B}_{H}^{{}_{} 2} {B}_{L}^{{}_{} 2} ;$$

$$\\ {t}_{a} = {n}_{0} + {n}_{1} {B}_{H} + {n}_{2} {B}_{L} + {n}_{3} {B}_{H} {B}_{L} + {n}_{4} {B}_{H}^{{}_{} 2} + {n}_{5} {B}_{L}^{{}_{} 2} + {n}_{6} {B}_{H}^{{}_{} 3} + {n}_{7} {B}_{L}^{{}_{} 3} + {n}_{8} {B}_{H}^{{}_{} 2} {B}_{L}^{{}_{} 2} ;$$

where B_{H} denotes target data collected by the high-energy bin of the ray emitting device and corrected by the method for data correction provided in the present disclosure, and B_{L} denotes target data collected by the low-energy bin of the ray emitting device and corrected by the method for data correction provided in the present disclosure. m₀, m₁, m₃ . . . denote decomposition coefficients of the polymethylmethacrylate homogeneous plate with the thickness of tₚ, and n₀, n₁, n₃ . . . denote decomposition coefficients of the homogeneous aluminum plate with the thickness of tₐ. The decomposition coefficients in the above equation may be determined based on the above equation and the corrected target data corresponding to the two homogeneous plates. Based on the above equations, the decomposition coefficients, and the target data of the subject to be detected, the distribution of the two base substances in the subject to be detected may be obtained.

Taking the homogeneous plate combination 1 as an example, for a ray emitting device having two energy bins (a high-energy bin H and a low-energy bin L), energies corresponding to the high-energy bin H and the low-energy bin L are different, e.g., the high-energy bin H may emit rays of 80 keV-140 keV, and the low-energy bin L may emit rays of 30 keV or less. B_{H} is the target data corrected by the method for data correction provided in the present disclosure for the data collected by the detector in response to the scanning of the homogeneous plate combination 1 by the high-energy bin of the ray emitting device. B_{L} is the target data corrected by the method for data correction provided in the present disclosure for the data collected by the detector in response to the scanning of the homogeneous plate combination 1 by the low-energy bin of the ray emitting device.

It should be understood that the processor may solve the above equation based on air correction data for at least nine homogeneous plate combinations to determine m₀, m₁, m₂, m₃, m₄, m₅, m₆, m₇, m₈, n₀, n₁, n₂, n₃, n₄, n₅, n₆, n₇, and n₈.

In 640, the distribution of base substances of the subject to be detected is determined based on the response data of the subject to be detected and the decomposition coefficient(s).

In some embodiments, the distribution determination module 240 may substitute the response data and the decomposition coefficient(s) of the subject to be detected into the relationship among the air correction data of the homogeneous plate combination, the thickness of each homogeneous plate of the homogeneous plate combination, and the decomposition coefficient corresponding to each base substance to determine the distribution of base substances in the subject to be detected. The response data of the subject to be detected may include air correction data of the detector in response to the scanning of the subject to be detected by the high-energy bin of the ray emitting device and air correction data of the detector in response to the scanning of the subject to be detected by the low-energy bin of the ray emitting device. More descriptions for obtaining the air correction data of the subject to be detected may be found in FIG. 3 and the related descriptions thereof, and will not be repeated herein.

Taking the above-mentioned polymethylmethacrylate homogeneous plate and homogeneous aluminum plate as an example, the subject to be detected may be composed of the polymethylmethacrylate homogeneous plate and the homogeneous aluminum plate, and the thickness tₓ₁ of the polymethylmethacrylate homogeneous plate and the thickness tₓ₂ of the homogeneous aluminum plate may be respectively expressed as the following equations:

$$\\ {t}_{x 1} = {m}_{0} + {m}_{1} {B}_{H , i} + {m}_{2} {B}_{L , i} + {m}_{3} {B}_{H , i} {B}_{L , i} + {m}_{4} {B}_{H , i}^{{}_{} 2} + {m}_{5} {B}_{L , i}^{{}_{} 2} + {m}_{6} {B}_{H , i}^{{}_{} 3} + {m}_{7} {B}_{L , i}^{{}_{} 3} + {m}_{8} {B}_{H , i}^{{}_{} 2} {B}_{L , i}^{{}_{} 2} ;$$

$$\\ {t}_{x 2} = {n}_{0} + {n}_{1} {B}_{H , i} + {n}_{2} {B}_{L , i} + {n}_{3} {B}_{H , i} {B}_{L , i} + {n}_{4} {B}_{H , i}^{{}_{} 2} + {n}_{5} {B}_{L , i}^{{}_{} 2} + {n}_{6} {B}_{H , i}^{{}_{} 3} + {n}_{7} {B}_{L , i}^{{}_{} 3} + {n}_{8} {B}_{H , i}^{{}_{} 2} {B}_{L , i}^{{}_{} 2} ;$$

where B_{H,i} denotes the air correction data of the detector in response to the scanning of the subject to be detected by the high-energy bin of the ray emitting device, and B_{L,i} denotes the air correction data of the detector in response to the scanning of the subject to be detected by the low-energy bin of the ray emitting device.

In some embodiments, by determining a plurality of homogeneous plate combinations, the processor may obtain air correction data of the detector in response to each homogeneous plate combination, and accurately determine the decomposition coefficients corresponding to different thicknesses of homogeneous plates for each material based on the air correction data of the detector in response to each homogeneous plate combination. Further, the processor may determine the distribution of base substances in the subject to be detected based on the response data of the subject to be detected and the decomposition coefficients.

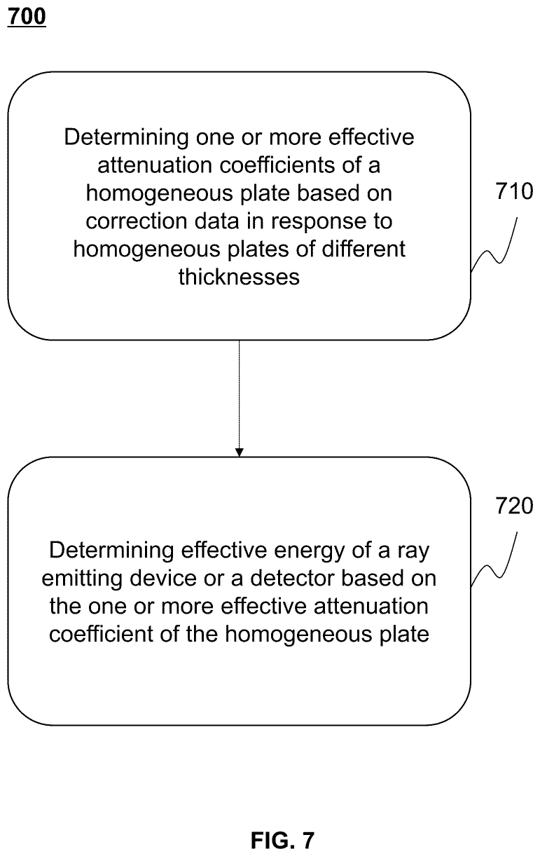

In some embodiments, the method for data correction may also be configured to determine an effective energy. FIG. 7 is a flowchart illustrating an exemplary process for determining effective energy according to some embodiments of the present disclosure. As shown in FIG. 7, process 700 may include the following operations.

In 710, an effective attenuation coefficient of the homogeneous plate is determined based on correction data in response to homogeneous plates of different thicknesses.

The effective attenuation coefficient may characterize a correspondence between the thickness of the homogeneous plate and the correction data. In some embodiments, the energy determination module 250 may determine a linear curve for characterizing the correspondence between the thickness of the homogeneous plate and the correction data based on the correction data for homogeneous plates of different thicknesses by linear fitting, and determine a slope of the linear curve as the effective attenuation coefficient of the homogeneous plate.

In 720, effective energy of the ray emitting device or the detector is determined based on the effective attenuation coefficient of the homogeneous plate.

The effective energy refers to the energy of rays emitted by the ray emitting device that are capable of being responded to by the detector after passing through the homogeneous plate. In some embodiments, the energy determination module 250 may determine the effective energy of the ray emitting device or detector based on the effective attenuation coefficient of the homogeneous plate. For example, the energy determination module 250 may determine the effective energy of the ray emitting device or detector based on the energy of the rays emitted by the ray emitting device and the effective attenuation coefficient of the homogeneous plate. Merely by way of example, the energy determination module 250 may determine a product of the energy of rays emitted by the ray emitting device and the effective attenuation coefficient as the effective energy.

In some embodiments, the effective attenuation coefficient of a homogeneous plate may be accurately determined based on the correction data of the detector in response to the homogeneous plates of different thicknesses. Then the effective energy of the ray emitting device or the detector may be quickly determined based on the effective attenuation coefficient of the homogeneous plate to provide a reference for parameterization of the medical scanning device.

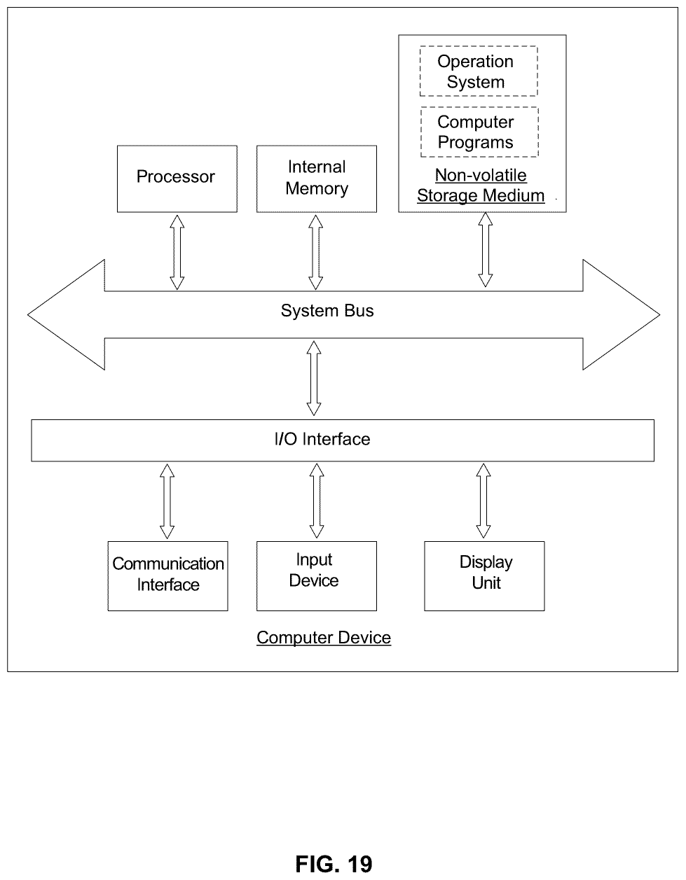

In one embodiment, a computer device is provided, which may be a terminal, and whose internal structure diagram may be shown in FIG. 19. The computer device may include a processor, a memory, a communication interface, a display, and an input device connected via a system bus. The processor of the computer device may be configured to provide computing and control capabilities. The memory of the computer device may include a non-volatile storage medium, an internal memory, etc. The non-volatile storage medium stores an operating system and one or more computer instructions. The internal memory provides an environment for the operation of the operating system and the computer instructions in the non-volatile storage medium. The communication interface of the computer device is configured to communicate with an external terminal in a wired or wireless manner. The wireless manner may be realized by WIFI, mobile cellular network, NFC (near field communication), or other technologies. The computer instructions may be executed by the processor to implement a method for data correction. The display of the computer device may be a liquid crystal display or an e-ink display. The input device of the computer device may be a touch layer covered on the display, a button, a trackball, or a touchpad provided on the housing of the computer device, an external keyboard, a touchpad, a mouse, or the like.

It should be appreciated by those skilled in the art that the structure illustrated in FIG. 19, which may be merely a block diagram of a portion of structures related to the present disclosure, does not constitute a limitation on the computer device where the present disclosure embodiment is applied, and the specific computer device may include more or fewer components than those shown in the drawings or may combine some of the components, or may have a different arrangement of components.

In one embodiment, a computer device including a memory and a processor is provided. The memory may store computer instructions, and the processor, when executing the computer instructions, realizes the following operations including obtaining three-dimensional coordinates of the plurality of detector pixel units, and determining the cosine correction data based on the three-dimensional coordinates of the plurality of detector pixel units and the coordinates of the focal point of the ray emitting device; determining response data to be corrected of the detector, and determining target data by correcting the response data to be corrected using the cosine correction data; and determining one or more correction coefficients corresponding to the plurality of detector pixel units based on the target data, and correcting response data of a subject to be detected based on the one or more correction coefficients.

In one embodiment, when executing the computer instructions, the processor may further implement the following operations including: for each row of the plurality of detector pixel units of the detector in a preset direction, determining a mean value of target data corresponding to the row of the plurality of detector pixel units, designating the mean value as correction data of the row of the plurality of detector pixel units; and determining the one or more correction coefficients based on correction data of all the plurality of detector pixel units.

In one embodiment, when executing the computer instructions, the processor may further implement the following operations including: obtaining a projection image of a testing phantom on the detector; determining a response center point of the detector based on the projection image; and determining three-dimensional coordinates of the plurality of detector pixel units based on the response center point, distances between the plurality of detector pixel units, and a distance between the focal point of the ray emitting device and the response center point.

In one embodiment, when executing the computer instructions, the processor may further implement the following operations including: for each of the plurality of detector pixel units, obtaining the cosine correction data by determining cosine of an angle between a first connecting line and a second connecting line according to the three-dimensional coordinates of the detector pixel unit and coordinates of the focal point. The first connecting line may be a connecting line between the detector pixel unit and the focal point of the ray emitting device, the second connecting line may be a connecting line between the focal point of the ray emitting device and a vertical point on the detector, and the second connecting line may be perpendicular to the detector.

In one embodiment, the vertical point is the response center point.

In one embodiment, when executing the computer instructions, the processor may further implement the following operations including obtaining phantom data and air data, wherein the phantom data is response data of the detector when a testing phantom is disposed between the ray emitting device and the detector, and the air data is response data of the detector when the testing phantom is not disposed between the ray emitting device and the detector; and determining the response data to be corrected based on the air data and phantom data.

In one embodiment, a non-transitory computer-readable storage medium is provided, on which computer instructions are stored. The computer instructions, when executed by the processor, may implement the following operations including obtaining three-dimensional coordinates of the plurality of detector pixel units, and determining the cosine correction data based on the three-dimensional coordinates of the plurality of detector pixel units and the coordinates of the focal point of the ray emitting device; determining response data to be corrected of the detector, and determining target data by correcting the response data to be corrected using the cosine correction data; and determining one or more correction coefficients corresponding to the plurality of detector pixel units based on the target data, and correcting response data of a subject to be detected based on the one or more correction coefficients.

In one embodiment, the computer instructions, when executed by the processor, may further implement the operations including: for each row of the plurality of detector pixel units of the detector in a preset direction, determining a mean value of target data corresponding to the row of the plurality of detector pixel units, designating the mean value as correction data of the row of the plurality of detector pixel units; and determining the one or more correction coefficients based on correction data of all the plurality of detector pixel units.

In one embodiment, the computer instructions, when executed by the processor, may further implement the operations including obtaining a projection image of a testing phantom on the detector; determining a response center point of the detector based on the projection image; and determining three-dimensional coordinates of the plurality of detector pixel units based on the response center point, distances between the plurality of detector pixel units, and a distance between the focal point of the ray emitting device and the response center point.

In one embodiment, the computer instructions, when executed by the processor, may further implement the operations including: for each of the plurality of detector pixel units, obtaining the cosine correction data by determining cosine of an angle between a first connecting line and a second connecting line according to the three-dimensional coordinates of the detector pixel unit and coordinates of the focal point. The first connecting line may be a connecting line between the detector pixel unit and the focal point of the ray emitting device, the second connecting line may be a connecting line between the focal point of the ray emitting device and a vertical point on the detector, and the second connecting line may be perpendicular to the detector.

In one embodiment, the second connecting line may be a line connecting the focal point of the ray emitting device and the response center point.

In one embodiment, the computer instructions, when executed by the processor, may further implement the operations including: obtaining phantom data and air data, wherein the phantom data is response data of the detector when a testing phantom is disposed between the ray emitting device and the detector, and the air data is response data of the detector when the testing phantom is not disposed between the ray emitting device and the detector; and determining the response data to be corrected based on the air data and phantom data.

In one embodiment, a computer instruction product including one or more computer instructions is provided. The computer instructions, when executed by the processor, may implement the operations including obtaining three-dimensional coordinates of the plurality of detector pixel units, and determining the cosine correction data based on the three-dimensional coordinates of the plurality of detector pixel units and the coordinates of the focal point of the ray emitting device; determining response data to be corrected of the detector, and determining target data by correcting the response data to be corrected using the cosine correction data; and determining one or more correction coefficients corresponding to the plurality of detector pixel units based on the target data, and correcting response data of a subject to be detected based on the one or more correction coefficients.

In one embodiment, the computer instructions, when executed by the processor, may further implement the operations including: for each row of the plurality of detector pixel units of the detector in a preset direction, determining a mean value of target data corresponding to the row of the plurality of detector pixel units, designating the mean value as correction data of the row of the plurality of detector pixel units; and determining the one or more correction coefficients based on correction data of all the plurality of detector pixel units.

In one embodiment, the computer instructions, when executed by the processor, may further implement the operations including obtaining a projection image of a testing phantom on the detector; determining a response center point of the detector based on the projection image; and determining three-dimensional coordinates of the plurality of detector pixel units based on the response center point, distances between the plurality of detector pixel units, and a distance between the focal point of the ray emitting device and the response center point.

In one embodiment, the computer instructions, when executed by the processor, may further implement the operations including: for each of the plurality of detector pixel units, obtaining the cosine correction data by determining cosine of an angle between a first connecting line and a second connecting line according to the three-dimensional coordinates of the detector pixel unit and coordinates of the focal point. The first connecting line may be a connecting line between the detector pixel unit and the focal point of the ray emitting device, the second connecting line may be a connecting line between the focal point of the ray emitting device and a vertical point on the detector, and the second connecting line may be perpendicular to the detector.

In one embodiment, the vertical point may be the response center point.

In one embodiment, the computer instructions, when executed by the processor, may further implement the operations including: obtaining phantom data and air data, wherein the phantom data is response data of the detector when a testing phantom is disposed between the ray emitting device and the detector, and the air data is response data of the detector when the testing phantom is not disposed between the ray emitting device and the detector; and determining the response data to be corrected based on the air data and phantom data.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended for those skilled in the art, though not expressly stated herein. These alterations, improvements, and modifications are intended to be suggested by this disclosure, and are within the spirit and scope of the exemplary embodiments of this disclosure.

Moreover, certain terminology has been used to describe embodiments of the present disclosure. For example, the terms “one embodiment,” “an embodiment,” and/or “some embodiments” mean that a particular feature, structure, or characteristic described in connection with the embodiment is included in at least one embodiment of the present disclosure. Therefore, it is emphasized and should be appreciated that two or more references to “an embodiment,” “one embodiment,” or “an alternative embodiment” in various portions of this specification are not necessarily all referring to the same embodiment. Furthermore, the particular features, structures, or characteristics may be combined as suitable in one or more embodiments of the present disclosure.

Furthermore, the recited order of processing elements or sequences, or the use of numbers, letters, or other designations thereof, are not intended to limit the claimed processes and methods to any order except as may be specified in the claims. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments. For example, although the implementation of various components described above may be embodied in a hardware device, it may also be implemented as a software-only solution, e.g., an installation on an existing server or mobile device.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. This method of disclosure, however, is not to be interpreted as reflecting an intention that the claimed subject matter requires more features than are expressly recited in each claim. Rather, claimed subject matter may lie in less than all features of a single foregoing disclosed embodiment.

In some embodiments, the numbers expressing quantities or properties used to describe and claim certain embodiments of the application are to be understood as being modified in some instances by the term “about,” “approximate,” or “substantially.” For example, “about,” “approximate,” or “substantially” may indicate ±20% variation of the value it describes, unless otherwise stated. Accordingly, in some embodiments, the numerical parameters set forth in the written description and attached claims are approximations that may vary depending upon the desired properties sought to be obtained by a particular embodiment. In some embodiments, the numerical parameters should be construed in light of the count of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

Each of the patents, patent applications, publications of patent applications, and other material, such as articles, books, specifications, publications, documents, things, and/or the like, referenced herein is hereby incorporated herein by this reference in its entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that may be employed may be within the scope of the application. Therefore, by way of example, but not of limitation, alternative configurations of the embodiments of the application may be utilized in accordance with the teachings herein. Accordingly, embodiments of the present application are not limited to that precisely as shown and described.

1. A method for data correction and applied to a medical scanning device, the medical scanning device including a ray emitting device and a detector, the detector including a plurality of detector pixel units, wherein the method comprises:
obtaining spatial positions of the plurality of detector pixel units;
determining cosine correction data based on the spatial positions of the plurality of detector pixel units and a spatial position of a focal point of the ray emitting device;
determining response data to be corrected of the detector;
determining target data by correcting the response data to be corrected using the cosine correction data;
determining one or more correction coefficients corresponding to the plurality of detector pixel units based on the target data; and
correcting response data of a subject to be detected based on the one or more correction coefficients.

2. The method of claim 1, wherein the determining one or more correction coefficients corresponding to the plurality of detector pixel units based on the target data includes:
for each row of the plurality of detector pixel units of the detector in a preset direction, determining a mean value of target data corresponding to the row of the plurality of detector pixel units,
designating the mean value as correction data of the row of the plurality of detector pixel units; and
determining the one or more correction coefficients based on correction data of all the plurality of detector pixel units.

3. The method of claim 2, wherein the response data to be corrected includes data of the detector in response to homogeneous plates of different thicknesses.

4. The method of claim 3, wherein the determining the one or more correction coefficients based on correction data of all the plurality of detector pixel units includes:
establishing a linear relationship between the target data and the correction data of the all the plurality of detector pixel units; and
determining the one or more correction coefficients by solving the linear relationship between the target data and the correction data of the all the plurality of detector pixel units based on the correction data of the all the plurality of detector pixel units corresponding to the homogeneous plates of different thicknesses.

5. The method of claim 1, wherein the obtaining spatial positions of the plurality of detector pixel units includes:
obtaining a projection image of a testing phantom on the detector;
determining a response center point of the detector based on the projection image; and
determining three-dimensional coordinates of the plurality of detector pixel units based on the response center point, distances between the plurality of detector pixel units, and a distance between the focal point of the ray emitting device and the response center point.

6. The method of claim 5, wherein the determining cosine correction data based on the spatial positions of the plurality of detector pixel units and a spatial position of a focal point of the ray emitting device includes:
for each of the plurality of detector pixel units, obtaining the cosine correction data by determining cosine of an angle between a first connecting line and a second connecting line according to the three-dimensional coordinates of the detector pixel unit and coordinates of the focal point, wherein
the first connecting line is a connecting line between the detector pixel unit and the focal point of the ray emitting device, the second connecting line is a connecting line between the focal point of the ray emitting device and a vertical point on the detector, and the second connecting line is perpendicular to the detector.

7. The method of claim 6, wherein the vertical point is the response center point.

8. The method of claim 1, wherein the determining response data to be corrected of the detector includes:
obtaining phantom data and air data by the detector, wherein
the phantom data is response data of the detector when a testing phantom is disposed between the ray emitting device and the detector, and
the air data is response data of the detector when the testing phantom is not disposed between the ray emitting device and the detector; and
determining the response data to be corrected based on the air data and the phantom data.

9. The method of claim 8, wherein the testing phantom includes homogeneous plates of at least two thicknesses.

10-18. (canceled)

19. A device for data correction including a processor, wherein the processor is configured to perform a method for data correction comprising:
obtaining spatial positions of the plurality of detector pixel units;
determining cosine correction data based on the spatial positions of the plurality of detector pixel units and a spatial position of a focal point of the ray emitting device;
determining response data to be corrected of the detector;
determining target data by correcting the response data to be corrected using the cosine correction data;
determining one or more correction coefficients corresponding to the plurality of detector pixel units based on the target data; and
correcting response data of a subject to be detected based on the one or more correction coefficients.

20. A non-transitory computer-readable storage medium for storing computer instructions, wherein the computer, when reads the computer instructions in the storage medium, performs a method for data correction comprising:
obtaining spatial positions of the plurality of detector pixel units;
determining cosine correction data based on the spatial positions of the plurality of detector pixel units and a spatial position of a focal point of the ray emitting device;
determining response data to be corrected of the detector;
determining target data by correcting the response data to be corrected using the cosine correction data;
determining one or more correction coefficients corresponding to the plurality of detector pixel units based on the target data; and
correcting response data of a subject to be detected based on the one or more correction coefficients.

21. The device of claim 19, wherein the determining one or more correction coefficients corresponding to the plurality of detector pixel units based on the target data includes:
for each row of the plurality of detector pixel units of the detector in a preset direction, determining a mean value of target data corresponding to the row of the plurality of detector pixel units,
designating the mean value as correction data of the row of the plurality of detector pixel units; and
determining the one or more correction coefficients based on correction data of all the plurality of detector pixel units.

22. The device of claim 21, wherein the response data to be corrected includes data of the detector in response to homogeneous plates of different thicknesses.

23. The device of claim 22, wherein the determining the one or more correction coefficients based on correction data of all the plurality of detector pixel units includes:
establishing a linear relationship between the target data and the correction data of the all the plurality of detector pixel units; and
determining the one or more correction coefficients by solving the linear relationship between the target data and the correction data of the all the plurality of detector pixel units based on the correction data of the all the plurality of detector pixel units corresponding to the homogeneous plates of different thicknesses.

24. The device of claim 19, wherein the obtaining spatial positions of the plurality of detector pixel units includes:
obtaining a projection image of a testing phantom on the detector;
determining a response center point of the detector based on the projection image; and
determining three-dimensional coordinates of the plurality of detector pixel units based on the response center point, distances between the plurality of detector pixel units, and a distance between the focal point of the ray emitting device and the response center point.

25. The device of claim 24, wherein the determining cosine correction data based on the spatial positions of the plurality of detector pixel units and a spatial position of a focal point of the ray emitting device includes:
for each of the plurality of detector pixel units, obtaining the cosine correction data by determining cosine of an angle between a first connecting line and a second connecting line according to the three-dimensional coordinates of the detector pixel unit and coordinates of the focal point, wherein
the first connecting line is a connecting line between the detector pixel unit and the focal point of the ray emitting device, the second connecting line is a connecting line between the focal point of the ray emitting device and a vertical point on the detector, and the second connecting line is perpendicular to the detector.

26. The device of claim 25, wherein the vertical point is the response center point.

27. The device of claim 19, wherein the determining response data to be corrected of the detector includes:
obtaining phantom data and air data by the detector, wherein
the phantom data is response data of the detector when a testing phantom is disposed between the ray emitting device and the detector, and
the air data is response data of the detector when the testing phantom is not disposed between the ray emitting device and the detector; and
determining the response data to be corrected based on the air data and the phantom data.

28. The device of claim 27, wherein the testing phantom includes homogeneous plates of at least two thicknesses.

29. The non-transitory computer-readable storage medium of claim 20, wherein the determining one or more correction coefficients corresponding to the plurality of detector pixel units based on the target data includes:
for each row of the plurality of detector pixel units of the detector in a preset direction, determining a mean value of target data corresponding to the row of the plurality of detector pixel units,
designating the mean value as correction data of the row of the plurality of detector pixel units; and
determining the one or more correction coefficients based on correction data of all the plurality of detector pixel units.
